(19) United States
(12) Patent Application Publication
(10) Pub. No.: US 2009/0227748 A1
(43) Pub. Date: Sep. 10, 2009

(54) Process for Preparing Olff in Polymers in the Presence of Catalyst Systems Having Photochromic Groups

(75) Inventor: Robert Larry Jones, Oakland, CA (US)

Correspondence Address: Basell USA Inc., Delaware Corporate Center II, 2 Righter Parkway, Suite #300, Wilmington, DE, 19803, US

(73) Assignee: BASELL POLYOLEFINE GMBH, Wesseling (DE)

(21) Appl. No.: 11/991,585

(22) PCT Filed: Aug. 31, 2006
(86) PCT No.: PCT/EP2006/008503

§ 371 Date: Mar. 6, 2008

Related U.S. Application Data
(60) Provisional application No. 60/725,412, filed on Oct. 11, 2005.

(30) Foreign Application Priority Data
Sep. 6, 2005 (DE) 10 2005 042 391.4

(51) Int. Cl. C08F 4/42; C08F 10/00; C08F 2/00; C07F 5/00; C07F 7/00; C07F 9/00; C07F 11/00; C07F 15/02; C07F 15/04; C07D 333/50; C07F 15/06; C07F 7/28

(52) U.S. Cl. 526/172; 526/348; 526/89; 526/220; 526/208; 526/204; 526/219.1; 526/90; 534/15; 556/51; 556/42; 556/57; 556/138; 556/136; 549/49; 502/103; 502/104

(57) ABSTRACT

The present invention relates to a process for preparing olefin polymers by polymerization or copolymerization of at least one olefin of the formula R^{a}—CH═CH—R^{b}, where R^{a} and R^{b} are identical or different and are each a hydrogen atom or a hydrocarbon radical having from 1 to 20 carbon atoms, or R^{a} and R^{b} together with the atoms connecting them can form a ring, at a temperature of from −60 to 200° C. and a pressure of from 0.5 to 100 bar, in solution, in suspension or in the gas phase in the presence of a catalyst system comprising one or more catalyst constituents, wherein the catalyst system comprises at least one photochromic group in one of the catalyst constituents, the corresponding catalyst systems, a process for preparing the catalyst systems of the invention, specific transition metal coordination compounds having photochromic groups, specific cyclopentadienyl derivatives and precursors thereof, in each case comprising a specific photochromic group, the use of the catalyst systems of the invention for the polymerization of olefins, the use of specific transition metal coordination compounds for preparing polymerization catalysts and polyolefins obtainable by the polymerization process of the invention.

This application is the U.S. national phase of International Application PCT/EP2006/008503, filed Aug. 31, 2006, claiming priority to German Patent Application 102005042391.4 filed Sep. 6, 2005, and the benefit under 35 U.S.C. 119(e) of U.S. Provisional Application No. 60/725,412, filed Oct. 11, 2005; the disclosures of International Application PCT/EP2006/008503, German Patent Application 102005042391.4 and U.S. Provisional Application No. 60/725,412, each as filed, are incorporated herein by reference.

### DESCRIPTION

The present invention relates to a process for preparing olefin polymers by polymerization or copolymerization of at least one olefin of the formula R^{a}—CH═CH—R^{b}, where R^{a} and R^{b} are identical or different and are each a hydrogen atom or a hydrocarbon radical having from 1 to 20 carbon atoms, or R^{a} and R^{b} together with the atoms connecting them can form a ring, at a temperature of from −60 to 200° C. and a pressure of from 0.5 to 100 bar, in solution, in suspension or in the gas phase in the presence of a catalyst system comprising one or more catalyst constituents, wherein the catalyst system comprises at least one photochromic group in one of the catalyst constituents.

In addition, the present invention relates to catalyst systems comprising one or more catalyst constituents, wherein the catalyst system comprises at least one photochromic group in one of the catalyst constituents, a process for preparing the catalyst systems of the invention, specific transition metal coordination compounds having photochromic groups, specific cyclopentadienyl derivatives and precursors thereof, in each case comprising a specific photochromic group, the use of the catalyst systems of the invention for the polymerization of olefins, the use of specific transition metal coordination compounds for preparing polymerization catalysts and polyolefins obtainable by the polymerization process of the invention.

Research and development on the use of transition metal coordination compounds, in particular metallocenes, as catalyst components for the polymerization and copolymerization of olefins with the objective of preparing tailored polyolefins has been pursued intensively in universities and in industry over the past 15 years. These new catalyst systems are frequently also referred to as “single site catalysts”.

The ethene-based polyolefins prepared by means of metallocene catalyst systems, in particular the propene-based polyolefins prepared by means of metallocene catalyst systems, now represent a dynamically growing market segment. Apart from metallocenes, new classes of transition metal coordination compounds which comprise no cyclopentadienyl ligands are now increasingly being examined as catalyst components. Examples are complexes of early transition metals which comprise phenoxyimine ligands (EP 874 005) or complexes of “late transition metals”, e.g. Ni, Pd (WO 96/23010), Fe or Co (WO 98/27124), which comprise uncharged ligands, for example diimines or bisiminopyridines.

Transition metal complexes comprising a photochromic group are known in principle. Angew. Chem. Int. Ed. 2005, 44, 2019-2021 describes a stereoselective cyclopropanation of styrene by means of ethyl diazoacetate in the presence of a copper-dithienylethene complex as catalyst.

The photochemical isomerization of bridged zirconocenes, in which an rac-meso isomerization takes place, is likewise known (Organometallics 1997, 16, 1724-1728). However, the ligand system itself considered in isolation is not changed in this process.

The process for preparing a particular polymer having defined structural features, for example tacticity, comonomer incorporation, molar mass or molar mass distribution, can be regulated and controlled via various process parameters. Suitable measures are, for example, choice of the catalyst, in particular the transition metal coordination compound as critical catalyst component, choice of the polymerization temperature, the monomer or comonomer concentration or the addition of regulators such as the molar mass regulator hydrogen.

To prepare reactor blends, i.e. mixtures of at least two different, defined polymers which are obtainable directly from one production plant, it is in principle possible to follow two routes. If one defined catalyst system, for example a “single site catalyst”, is employed, the catalyst system has to be subjected to at least two defined different reaction conditions during its residence time in the polymerization process, as can be achieved, for example, in a reactor cascade having at least two reactors connected in series or in a multizone reactor, with defined zones having different reaction conditions being present in the latter type of reactor.

A further possibility which is discussed is the use of at least two catalyst systems which due to their nature produce different polymers under identical polymerization conditions.

The combination of the two concepts, i.e. the use of different reaction conditions and the use of different catalyst systems, offers further opportunities for producing reactor blends in a targeted manner.

It was therefore an object of the present invention to discover a process for preparing polyolefins, in which a further means of controlling the polymerization process in a targeted manner in order to be able to prepare tailored polyolefins independently of the control possibilities already known is provided.

We have accordingly found the process for preparing olefin polymers which has been mentioned at the outset and is defined in the claims and in which catalyst systems comprising one or more catalyst components of which at least one comprises at least one photochromic group are used. Furthermore, we have found the corresponding catalyst systems and a process for preparing them, specific transition metal coordination compounds and specific cyclopentadienyl derivatives which each comprise photochromic groups, and also polyolefins which can be obtained by the polymerization process of the invention.

For the purposes of the present invention, the term photochromism is a reversible transformation brought about by visible or ultraviolet light of a substance into another substance whose color (adsorption spectrum) differs from the starting compound. The reverse reaction can be triggered by light of a different wavelength or by heat or can occur spontaneously. In the case of organic compounds, the energy required for the reversible transformation of the organic photochromic group, i.e. light of a particular wavelength, depends on the respective photochromic system and its specific substitution pattern. Examples and applications of photochromic systems are described in Chem. Rev. 2000, 100, 1685-1890.

In the process of the invention, at least one olefin of the formula R^{a}—CH═CH—R^{b}, where R^{a} and R^{b} are identical or different and are each a hydrogen atom or a hydrocarbon radical having from 1 to 20 carbon atoms, in particular from 1 to 10 carbon atoms, or R^{a} and R^{b} together with the atoms connecting them can form one or more rings, is polymerized.

Examples of such olefins are 1-olefins having 2 to 20, preferably from 2 to 10, carbon atoms, e.g. ethene, propene, 1-butene, 1-pentene, 1-hexene, 1-heptene, 1-octene, 1-decene or 4-methyl-1-pentene, or unsubstituted or substituted vinylaromatic compounds such as styrene and styrene derivatives, or dienes such as 1,3-butadiene, 1,4-hexadiene, 1,7-octadiene, 5-ethylidene-2-norbornene, norbornadiene, ethylnorbornadiene or cyclic olefins such as norbornene, tetracyclododecene or methylnorbornene.

In the process of the invention, preference is given to homopolymerizing ethene, propene or 1-butene or copolymerizing one of these together with a further comonomer such as ethene, propene, 1-butene, 1-pentene, 1-hexene, 4-methyl-1-pentene and/or 1-octene and/or cyclic olefins such as norbornene and/or dienes having from 4 to 20 carbon atoms, e.g. 1,4-hexadiene, norbornadiene, ethylidenenorbornene or ethylnorbornadiene. In particular, propene is copolymerized with ethene, ethene is copolymerized with butene or hexene and butene is copolymerized with propylene and/or ethene. The process of the invention is very particularly useful for preparing elastomers or plastomers, for example the polypropylene plastomers described in Macromol. Chem. Phys. 2005, 206, 1231-1240.

The process of the invention can be carried out at temperatures in the range from −60 to 200° C., preferably in the range from 0 to 150° C., in particular from 50 to 100° C.

The process of the invention can be carried out at a pressure of from 0.5 to 100 bar, preferably from 5 to 65 bar.

The process of the invention can be carried out in a known manner in solution, in suspension or in the gas phase in the customary reactors used for the polymerization of olefins. The process can be carried out continuously or batchwise, preferably continuously, in one or more stages. As solvents or suspension media, it is possible to use inert hydrocarbons, for example propane, isobutane or hexane, or else the monomers themselves, for example propene. The process can, for example, be carried out in a reactor cascade in which the polymerization is carried out in a solvent or suspension medium, preferably in propene, in a first polymerization step and the reaction, in particular a copolymerization, is carried out in the gas phase in a second polymerization step.

The mean residence times in the process of the invention are usually from 0.5 to 5 hours, preferably from 0.5 to 3 hours.

The process of the invention is carried out in the presence of a catalyst system comprising one or more catalyst constituents, with the catalyst system comprising at least one photochromic group in one of the catalyst constituents.

In the case of classical Ziegler-Natta catalysts which are suitable for the preparation of highly isotactic polypropylenes, the photochromic group can, for example, be bound to the known internal and external donor compounds, for example to diether derivatives, succinate derivatives or benzoate derivatives.

In the case of “single site catalysts”, which usually comprise at least one transition metal coordination compound and at least one cocatalyst, the photochromic group can be part of a transition metal coordination compound or of a cocatalyst. In the transition metal coordination compounds, the photochromic group is preferably bound to a ligand of the transition metal coordination compound, with the photochromic group itself preferably not coordinating to the transition metal center.

The photochromic group used in the process of the invention is usually an organic, i.e. carbon-containing, group, preferably selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives, in particular selected from the group consisting of 1,2-diarylethene derivatives, 1,2-diheteroarylethene derivatives and dihydropyrene derivatives and particularly preferably selected from among 1,2-di(3-thienyl)ethene derivatives.

The isomerization of the photochromic group brought about by light can in principle occur prior to the polymerization reaction or during the polymerization reaction.

The light used for the isomerization of the photochromic group usually has a wavelength in the range from 150 nm to 1000 nm, in particular from 180 nm to 800 nm.

As light source, it is in principle possible to use all known light-producing systems such as incandescent lamps, mercury vapor lamps, light-emitting diodes or the various types of laser. The choice of the light source depends on the energy required for the transformation of the photochromic group.

The process of the invention is preferably carried out for at least part of the time in the presence of at least one light source which is suitable for transforming a photochromic group from the state A into the state B or from the state B into the state A.

This transformation from state A into the state B and back can be illustrated schematically using the case of the diarylethene skeleton as an example.

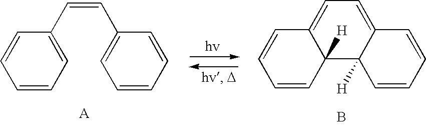

The above-described process of the invention is preferably carried out in the presence of a catalyst system comprising at least one transition metal coordination compound and, if appropriate, a cocatalyst, with the transition metal coordination compound comprising at least one ligand which is substituted by or fused with at least one photochromic group. If the transition metal coordination compound comprises, for example, two different photochromic groups which can be isomerized by light of differing wavelength, further possible ways of controlling the polymerization process in a targeted manner are opened up in the process of the invention.

As transition metal coordination compound in the polymerization process of the invention, it is in principle possible to use all known classes of polymerization-active transition metal compounds, in particular after preactivation by means of suitable cocatalysts, if one of the ligands of the transition metal center can comprise a photochromic group. Various classes of transition metal coordination compounds which can be used as catalyst component for preparing polymerization catalysts are described in Chem. Rev. 2000, Vol. 100, No. 4.

In the process of the invention, preference is given to a transition metal coordination compound which comprises at least one ligand selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms which is bound to the transition metal center and is substituted by or fused with at least one photochromic group.

Preference is likewise given in the process of the invention to a transition metal coordination compound in which the transition metal is selected from the group of elements consisting of Sc, Yb, La, Ti, Zr, Hf, V, Nb, Ta, Cr, Mo, W, Fe, Co, Ni, Pd and the lanthanides, preferably Ti, Zr, Hf, Cr and Fe, particularly preferably Zr, Hf, Cr and Fe, in particular Zr and Hf, and which comprises at least one ligand comprising at least one photochromic group, with the ligand preferably being selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms, preferably cyclopentadienyl derivatives and uncharged, tridentate nitrogen ligands having three coordinating nitrogen atoms, in particular cyclopentadienyl derivatives.

Illustrative examples which do not, however, restrict the scope of the invention of transition metal coordination compounds which can be used in the process of the invention are:

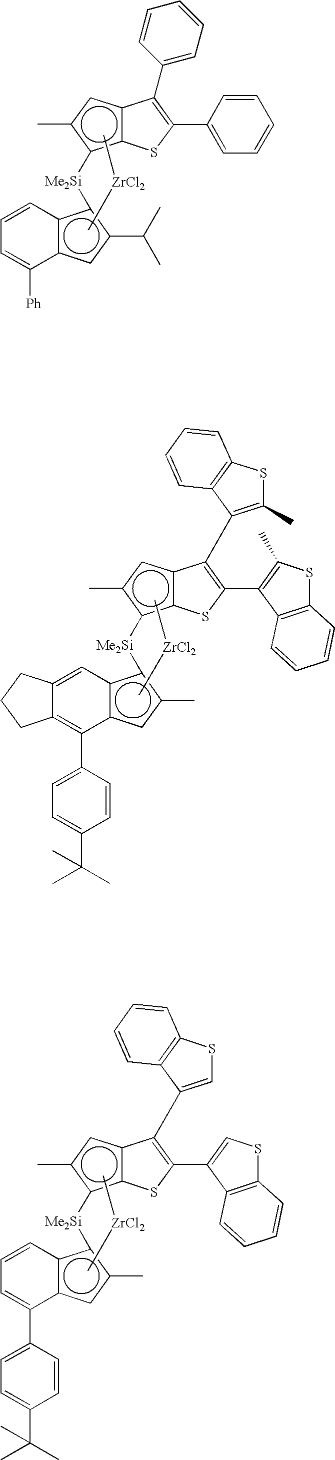

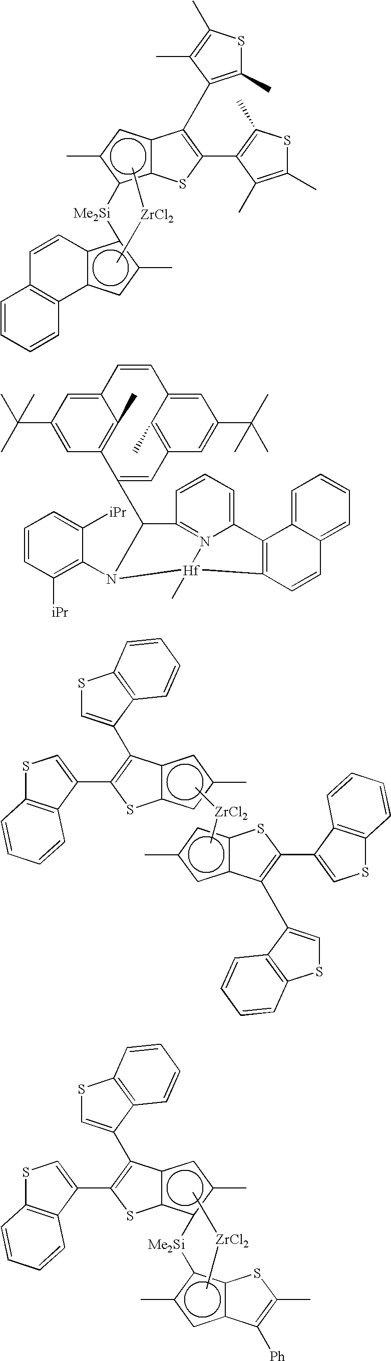

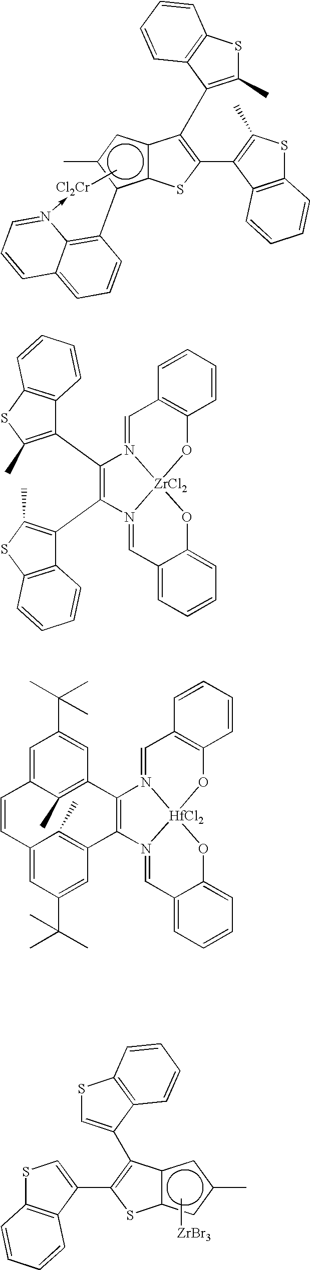

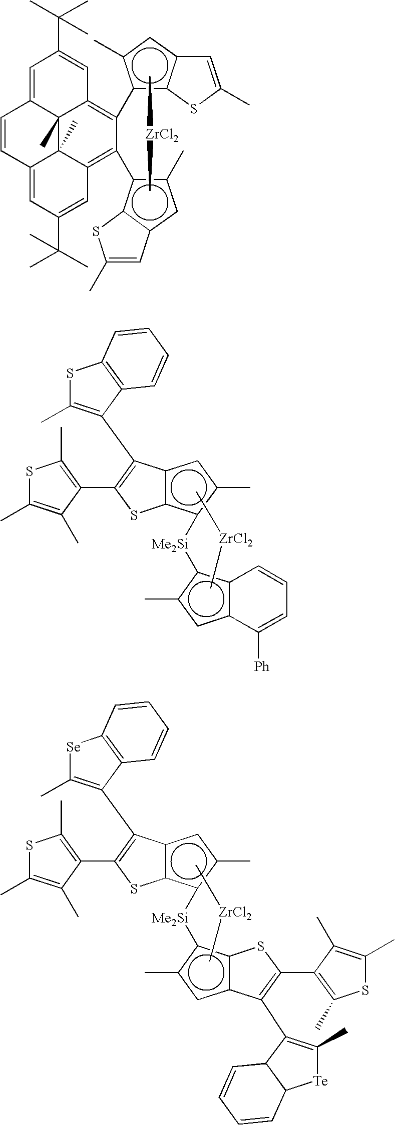

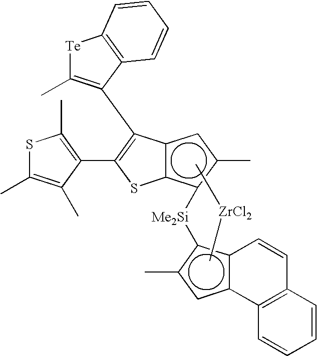

The invention further provides a transition metal coordination compound which can preferably be used as catalyst component in the process of the invention. In this transition metal coordination compound, the transition metal is selected from the group of elements consisting of Sc, Yb, La, Ti, Zr, Hf, V, Nb, Ta, Mo, W, Fe, Co, Ni, Pd and the lanthanides, preferably Ti, Zr, Hf and Fe, particularly preferably Zr, Hf and Fe, in particular Zr and Hf, and the transition metal coordination compound comprises at least one ligand comprising at least one photochromic group. In this inventive transition metal coordination compound, the photochromic group is preferably selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives, in particular 1,2-diarylethene derivatives, 1,2-diheteroarylethene derivatives and dihydropyrene derivatives and very particularly preferably 1,2-di(3-thienyl)ethene derivatives.

The invention likewise provides a transition metal coordination compound in which the transition metal is selected from the group of the elements consisting of Sc, Yb, La, Ti, Zr, Hf, V, Nb, Ta, Cr, Mo, W, Fe, Co, Ni, Pd and the lanthanides, preferably Ti, Zr, Hf, Cr and Fe, particularly preferably Zr, Hf and Fe, in particular Zr and Hf, and which comprises at least one ligand selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms, preferably cyclopentadienyl derivatives and uncharged, tridentate nitrogen ligands having three coordinating nitrogen atoms, in particular cyclopentadienyl derivatives, which is substituted by or fused with at least one photochromic group. This transition metal coordination compound, too, can preferably be used as catalyst component in the process of the invention.

Preference is given to transition metal coordination compounds of the formula (I)

Z¹AₙZ²ₚMxₘ (I)

where
Z¹ is a ligand selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms, and Z¹, comprises a photochromic group PCG¹ selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives,
A is a bridge between the ligands Z¹ and Z² which consists of a divalent atom or a divalent group,
Z² is identical to or different from Z¹ and is selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms, with Z² optionally being able to comprise a photochromic group PCG² selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives,
p is zero or 1,
n is zero or 1,
M is a transition metal selected from the group of elements consisting of Sc, Yb, La, Ti, Zr, Hf, V, Nb, Ta, Cr, Mo, W, Fe, Co, Ni, Pd and the lanthanides,
the radicals X are identical or different and are each an organic or inorganic radical, with two radicals X also being able to be joined to one another, and
m is 0, 1, 2 or 3.

Z¹ is a ligand selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms, preferably cyclopentadienyl derivatives or uncharged, tridentate nitrogen ligands having three coordinating nitrogen atoms, in particular cyclopentadienyl derivatives, with Z¹ comprising a photochromic group PCG¹ selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives, preferably 1,2-diarylethene derivatives, 1,2-diheteroarylethene derivatives and dihydropyrene derivatives and very particularly preferably 1,2-di(3-thienyl)ethene derivatives.

Z¹ is preferably a cyclopentadienyl ligand of the formula (II) or an isomeric form thereof which is produced by irradiation with visible or ultraviolet light and has the formula (IIa)

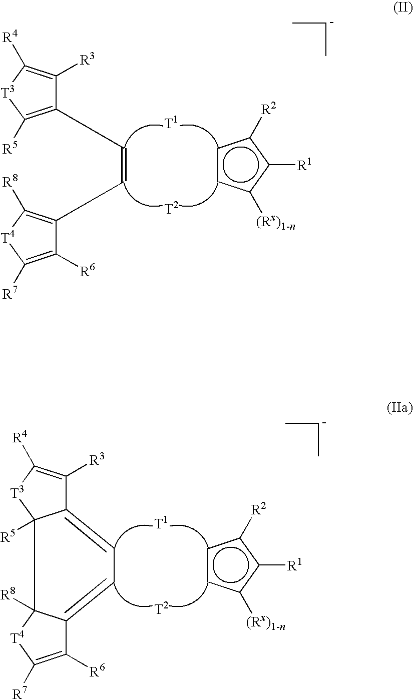

where
R¹ is hydrogen or an organic radical having from 1 to 40 carbon atoms,
R² is hydrogen or an organic radical having from 1 to 40 carbon atoms, in particular hydrogen,
R³, R⁴, R⁶, R⁷ are identical or different and are each hydrogen, halogen or an organic radical having from 1 to 40 carbon atoms or two adjacent radicals R³ and R⁴ and/or R⁶ and R⁷ together with the atoms connecting them form a monocyclic or polycyclic, substituted or unsubstituted, aliphatic or aromatic ring system which has from 4 to 40 carbon atoms and can also comprise heteroatoms selected from the group consisting of the elements Si, Ge, N, P, O, S, Se and Te,
R⁵, R⁸ are identical or different, preferably identical, and are each hydrogen or an organic radical having from 1 to 40 carbon atoms,
R^{x} is hydrogen or an organic radical having from 1 to 40 carbon atoms,
n is as defined in formula (I), i.e. zero in the case of unbridged ligands Z¹ and 1 in the case of a ligand Z¹ linked to A,
T¹ is a single bond or a divalent organic group having from 1 to 40 carbon atoms,
T² is O, S, Se, Te, NR⁹, PR⁹ or CR⁹₂, where the radicals R⁹ are identical or different and are each hydrogen or an organic radical having from 1 to 40 carbon atoms, and
T³, T⁴ are identical or different and are each O, S, Se, Te, NR⁹ or PR⁹.

The substituents according to the present invention are, unless restricted further, defined as follows:

The term “organic radical having from 1 to 40 carbon atoms” as used in the present text refers to, for example, C₁-C₄₀-alkyl radicals, C₁-C₁₀-fluoroalkyl radicals, C₁-C₁₂-alkoxy radicals, saturated C₃-C₂₀-heterocyclic radicals, C₆-C₄₀-aryl radicals, C₂-C₄₀-heteroaromatic radicals, C₆-C₁₀-fluoroaryl radicals, C₆-C₁₀-aryloxy radicals, silyl radicals having from 3 to 24 carbon atoms, C₂-C₂₀-alkenyl radicals, C₂-C₂₀-alkynyl radicals, C₇-C₄₀-arylalkyl radicals or C₈-C₄₀-arylalkenyl radicals. An organic radical is in each case derived from an organic compound. Thus, the organic compound methanol can in principle give rise to three different organic radicals having one carbon atom, namely methyl (H₃C—), methoxy (H₃C—O—) and hydroxymethyl (HOC(H₂)—).

The term “alkyl” as used in the present text encompasses linear or singly or multiply branched saturated hydrocarbons which can also be cyclic. Preference is given to a C₁-C₁₈-alkyl radical such as methyl, ethyl, n-propyl, n-butyl, n-pentyl, n-hexyl, n-heptyl, n-octyl, n-nonyl, n-decyl, cyclopentyl, cyclohexyl, isopropyl, isobutyl, isopentyl, isohexyl, sec-butyl or tert-butyl.

The term “alkenyl” as used in the present text encompasses linear or singly or multiply branched hydrocarbons having one or more C—C double bonds which can be cumulated or alternating.

The term “saturated heterocyclic radical” as used in the present text refers to, for example, monocyclic or polycyclic, substituted or unsubstituted aliphatic or aromatic hydrocarbon radicals in which one or more carbon atoms, CH groups and/or CH₂ groups have been replaced by heteroatoms which are preferably selected from the group consisting of the elements O, S, N and P. Preferred examples of substituted or unsubstituted saturated heterocyclic radicals are pyrrolidinyl, imidazolidinyl, pyrazolidinyl, piperidyl, piperazinyl, morpholinyl, tetrahydrofuranyl, tetrahydropyranyl, tetrahydrothiophenyl and the like, and also methyl-, ethyl-, propyl-, isopropyl- and tert-butyl-substituted derivatives thereof.

The term “aryl” as used in the present text refers to, for example, aromatic and optionally fused polyaromatic hydrocarbon radicals which may be monosubstituted or polysubstituted by linear or branched C₁-C₁₈-alkyl, C₁-C₁₈-alkoxy, C₂-C₁₀-alkenyl or halogen, in particular fluorine. Preferred examples of substituted and unsubstituted aryl radicals are, in particular, phenyl, pentafluorophenyl, 4-methylphenyl, 4-ethylphenyl, 4-n-propylphenyl, 4-isopropylphenyl, 4-tert-butylphenyl, 4-methoxyphenyl, 1-naphthyl, 9-anthryl, 9-phenanthryl, 3,5-dimethylphenyl, 3,5-di-tert-butylphenyl or 4-trifluoromethylphenyl.

The term “heteroaromatic radical” as used in the present text refers to, for example, aromatic hydrocarbon radicals in which one or more carbon atoms have been replaced by nitrogen, phosphorus, oxygen or sulfur atoms or combinations thereof. These may, like the aryl radicals, optionally be monosubstituted or polysubstituted by linear or branched C₁-C₁₈-alkyl, C₂-C₁₀-alkenyl or halogen, in particular fluorine. Preferred examples are furyl, thienyl, pyrrolyl, pyridyl, pyrazolyl, imidazolyl, oxazolyl, thiazolyl, pyrimidinyl, pyrazinyl and the like, and also methyl-, ethyl-, propyl-, isopropyl- and tert-butyl-substituted derivatives thereof.

The term “arylalkyl” as used in the present text refers to, for example, aryl-comprising substituents whose aryl radical is linked via an alkyl chain to the remainder of the molecule. Preferred examples are benzyl, substituted benzyl, phenethyl, substituted phenethyl and the like.

The terms fluoroalkyl and fluoroaryl mean that at least one hydrogen atom, preferably more than one and at most all hydrogen atoms, of the corresponding radical have been replaced by fluorine atoms. Examples of preferred fluorine-comprising radicals are trifluoromethyl, 2,2,2-trifluoroethyl, pentafluorophenyl, 4-trifluoromethylphenyl, 4-perfluoro-tert-butylphenyl and the like.

In particular, the substituents on the ligand Z¹ of the formula (II) or (IIa) are defined as follows.

The radical R¹ is hydrogen or an organic radical having from 1 to 40 carbon atoms, for example C₁-C₄₀-alkyl, C₁-C₁₀-fluoroalkyl, C₂-C₄₀-alkenyl, C₆-C₄₀-aryl, C₆-C₁₀-fluoroaryl, arylalkyl, arylalkenyl or alkylaryl having from 1 to 10, preferably from 1 to 4, carbon atoms in the alkyl radical and from 6 to 22, preferably from 6 to 10, carbon atoms in the aryl radical, a saturated heterocycle having from 2 to 40 carbon atoms or a C₂-C₄₀-heteroaromatic radical having at least one heteroatom selected from the group consisting of the elements O, N, S, P and Se, in particular O, N and S, with the heteroaromatic radical being able to be substituted by further radicals R¹⁰, where R¹⁰ is an organic radical having from 1 to 20 carbon atoms, for example C₁-C₁₀-, preferably C₁-C₄-alkyl, C₆-C₁₅-, preferably C₆-C₁₀-aryl, alkylaryl, arylalkyl, fluoroalkyl or fluoroaryl each having from 1 to 10, preferably from 1 to 4, carbon atoms in the alkyl radical and from 6 to 18, preferably from 6 to 10, carbon atoms in the aryl radical, and a plurality of radicals R¹⁰ can be identical or different. R¹ is preferably a linear C₁-C₁₂-, preferably C₁-C₄-alkyl radical, a branched C₃-C₁₂-, preferably C₃-C₆-alkyl radical, a C₄-C₁₀-, preferably C₅-C₈-cycloalkyl radical, a substituted furyl radical or a substituted thienyl radical. The radical R¹ is particularly preferably hydrogen, methyl, ethyl, n-propyl, i-propyl, n-butyl or cyclohexyl.

The radical R² is hydrogen or an organic radical having from 1 to 40 carbon atoms, for example C₁-C₄₀-alkyl, C₁-C₁₀-fluoroalkyl, C₂-C₄₀-alkenyl, C₆-C₄₀-aryl, C₆-C₁₀-fluoroaryl, arylalkyl, arylalkenyl or alkylaryl having from 1 to 10, preferably from 1 to 4, carbon atoms in the alkyl radical and from 6 to 22, preferably from 6 to 10, carbon atoms in the aryl radical, a saturated heterocycle having from 2 to 40 carbon atoms or a C₂-C₄₀-heteroaromatic radical having at least one heteroatom selected from the group consisting of the elements O, N, S, P and Se, in particular O, N and S, with the heteroaromatic radical being able to be substituted by further radicals R¹⁰. R² is preferably hydrogen.

The radicals R³, R⁴, R⁶ and R⁷ are identical or different and are each hydrogen, halogen such as fluorine, chlorine, bromine or iodine, preferably fluorine, or an organic radical having from 1 to 40 carbon atoms, for example a cyclic, branched or unbranched C₁-C₂₀-, preferably C₁-C₈-alkyl radical, a C₂-C₂₀-, preferably C₂-C₈-alkenyl radical, a C₆-C₂₂-, preferably C₆-C₁₀-aryl radical, an alkylaryl or arylalkyl radical having from 1 to 10, preferably from 1 to 4, carbon atoms in the alkyl radical and from 6 to 22, preferably from 6 to 10, carbon atoms in the aryl radical, where the radicals may also be halogenated, a saturated heterocycle having from 2 to 40 carbon atoms or a C₂-C₄₀-heteroaromatic radical having at least one heteroatom selected from the group consisting of elements O, N, S, P and Se, in particular O, N and S, with the heteroaromatic radical being able to be substituted by further radicals R¹⁰, or two adjacent radicals R³ and R⁴ and/or R⁶ and R⁷ together with the atoms connecting them form a monocyclic or polycyclic, substituted or unsubstituted aliphatic or aromatic ring system which has from 4 to 40 carbon atoms and can also comprise heteroatoms selected from the group consisting of the elements Si, Ge, N, P, O, S, Se and Te, in particular N and S. The radicals R³ and R⁴ or R⁶ and R⁷ together with the atoms connecting them preferably in each case form a substituted or unsubstituted, in particular unsubstituted, 1,3-butadiene-1,4-diyl group.

The radicals R⁵ and R⁸ are identical or different, preferably identical, and are each hydrogen or an organic radical having from 1 to 40 carbon atoms, for example a cyclic, branched or unbranched C₁-C₂₀-, preferably C₁-C₈-alkyl radical, a C₁-C₁₀-, preferably C₁-C₄-alkoxy radical, a C₂-C₂₀-, preferably C₂-C₈-alkenyl radical, a C₆-C₂₂-, preferably C₆-C₁₀-aryl radical, an alkylaryl or arylalkyl radical having from 1 to 10, preferably from 1 to 4, carbon atoms in the alkyl radical and from 6 to 22, preferably from 6 to 10, carbon atoms in the aryl radical, with the radicals also being able to be halogenated, a saturated heterocycle having from 2 to 40 carbon atoms or a C₂-C₄₀-heteroaromatic radical having at least one heteroatom selected from the group consisting of the elements O, N, S, P and Se, in particular O, N and S, with the heteroaromatic radical being able to be substituted by further radicals R¹⁰. R⁵ and R⁸ are preferably each hydrogen, methyl, hexyl, trifluoromethyl, isopropyl, methoxy or ethoxy, in particular methyl.

The radical R^{x} is hydrogen or an organic radical having from 1 to 40 carbon atoms, for example C₁-C₄₀-alkyl, C₁-C₁₀-fluoroalkyl, C₂-C₄₀-alkenyl, C₆-C₄₀-aryl, C₆-C₁₀-fluoroaryl, arylalkyl, arylalkenyl or alkylaryl having from 1 to 10, preferably from 1 to 4, carbon atoms in the alkyl radical and from 6 to 22, preferably from 6 to 10, carbon atoms in the aryl radical, a saturated heterocycle having from 2 to 40 carbon atoms or a C₂-C₄₀-heteroaromatic radical having at least one heteroatom selected from the group consisting of the elements O, N, S, P and Se, in particular O, N and S, with the heteroaromatic radical being able to be substituted by further radicals R¹⁰. R^{x} is preferably hydrogen, a linear C₁-C₁₂-, preferably C₁-C₄-alkyl radical, a branched C₃-C₁₂-, preferably C₃-C₆-alkyl radical, a C₄-C₁₀-, preferably C₅-C₈-cycloalkyl radical, a substituted or alkyl-substituted C₆-C₁₄-aryl radical, a substituted furyl radical or a substituted thienyl radical such as 2-(5-methyl)thienyl. The radical R^{x} is particularly preferably hydrogen, methyl, ethyl, n-propyl, i-propyl, n-butyl or cyclohexyl.

T¹ is a single bond or a divalent organic group having from 1 to 40 carbon atoms. T¹ is preferably a single bond, a divalent radical CR¹⁰₂ or a divalent radical CR¹⁰₂—CR¹⁰₂, where the radicals R¹⁰ may be identical or different and are as defined above. T¹ is particularly preferably a single bond.

T² is O, S, Se, Te, NR⁹, PR⁹ or CR⁹₂, where the radicals R⁹ are identical or different and are each hydrogen or an organic radical having from 1 to 40 carbon atoms, for example a cyclic, branched or unbranched C₁-C₂₀-, preferably C₁-C₈-alkyl radical, a C₂-C₂₀-, preferably C₂-C₈-alkenyl radical, a C₆-C₂₂-, preferably C₆-C₁₀-aryl radical, an alkylaryl or arylalkyl radical having from 1 to 10, preferably from 1 to 4, carbon atoms in the alkyl radical and from 6 to 22, preferably from 6 to 10, carbon atoms in the aryl radical, with the radicals also being able to be halogenated, a saturated heterocycle having from 2 to 40 carbon atoms or a C₂-C₄₀-heteroaromatic radical having at least one heteroatom selected from the group consisting of the elements O, N, S, P and Se, in particular O, N and S, with the heteroaromatic radical being able to be substituted by further radicals R¹⁰. T² is preferably S.

T³, T⁴ are identical or different and are each O, S, Se, Te, NR⁹ or PR⁹, where R⁹ is as defined above. Preference is given to T³ and T⁴ being identical or different, preferably identical, and each being S, Se or NR⁹, in particular S.

A is a bridge between the ligands Z¹ and Z² which consists of a divalent atom or a divalent group. Examples of A are:

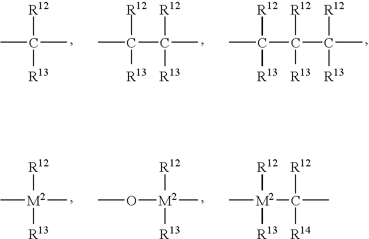

—B(R¹²)—, —B(NR¹²R¹³)—, —Al(R¹²)—, —O—S—, —S(O)—, —S((O)₂)—, —N(R¹²)—, —C(O)—, —P(R¹²)— or —P(O) (R¹²)—,
   in particular

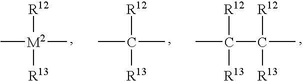

where

M² is silicon, germanium or tin, preferably silicon or germanium, particularly preferably silicon, and R¹², R¹³ and R¹⁴ are identical or different and are each a hydrogen atom, a halogen atom, a trimethylsilyl group, a C₁-C₁₀, preferably C₁-C₃-alkyl group, a C₁-C₁₀-fluoroalkyl group, a C₆-C₁₀-fluoroaryl group, a C₆-C₁₀-aryl group, a C₁-C₁₀-, preferably C₁-C₃-alkoxy group, a C₇-C₁₅-alkylaryloxy group, a C₂-C₁₀-, preferably C₂-C₄-alkenyl group, a C₇-C₄₀-arylalkyl group, a C₈-C₄₀-arylalkenyl group or a C₇-C₄₀-alkylaryl group, or two adjacent radicals together with the atoms connecting them form a saturated or unsaturated ring having from 4 to 15 carbon atoms.

Preferred embodiments of A are the bridges:

dimethylsilanediyl, methylphenylsilanediyl, diphenylsilanediyl, methyl-tert-butylsilanediyl, dimethylgermanediyl, ethylidene, 1-methylethylidene, 1,1-dimethylethylidene, 1,2-dimethylethylidene, 1,1,2,2-tetramethylethylidene, dimethylmethylidene, phenylmethylmethylidene and diphenylmethylidene, in particular dimethylsilanediyl, diphenylsilanediyl and ethylidene.

A is particularly preferably a substituted silylene group or a substituted or unsubstituted ethylene group, preferably a substituted silylene group such as dimethylsilanediyl, methylphenylsilanediyl, methyl-tert-butylsilanediyl or diphenylsilanediyl, in particular dimethylsilanediyl.

Z² is identical to or different from Z¹ and is selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms, with Z² optionally being able to comprise a photochromic group PCG² selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives, preferably 1,2-diarylethene derivatives, 1,2-diheteroarylethene derivatives and dihydropyrene derivatives and very particularly preferably 1,2-di(3-thienyl)ethene derivatives.

p is zero or 1, preferably 1.

n is zero or 1, preferably 1.

M is a transition metal selected from the group of elements consisting of Sc, Yb, La, Ti, Zr, Hf, V, Nb, Ta, Cr, Mo, W, Fe, Co, Ni, Pd and the lanthanides, preferably Ti, Zr, Hf, Cr and Fe, particularly preferably Zr, Hf and Fe, in particular Zr and Hf.

The radicals X are identical or different, preferably identical, and are each an organic or inorganic radical, with two radicals X also being able to be joined to one another. X is preferably halogen, for example fluorine, chlorine, bromine, iodine, preferably chlorine, hydrogen, C₁-C₂₀-, preferably C₁-C₄-alkyl, in particular methyl, C₂-C₂₀-, preferably C₂-C₄-alkenyl, C₆-C₂₂-, preferably C₆-C₁₀-aryl, an alkylaryl or arylalkyl group having from 1 to 10, preferably from 1 to 4, carbon atoms in the alkyl radical and from 6 to 22, preferably from 6 to 10, carbon atoms in the aryl radical, —OR¹¹ or —NR¹¹R^{11a}, with two radicals X, preferably two radicals —OR¹¹ also being able to be joined to one another. It is also possible for two radicals X to form a substituted or unsubstituted diene ligand, in particular a 1,3-diene ligand. The radicals R¹¹ and R^{11a} are each C₁-C₁₀-, preferably C₁-C₄-alkyl, C₆-C₁₅-, preferably C₆-C₁₀-aryl, alkylaryl, arylalkyl, fluoroalkyl or fluoroaryl each having from 1 to 10, preferably from 1 to 4, carbon atoms in the alkyl radical and from 6 to 22, preferably from 6 to 10, carbon atoms in the aryl radical. X is very particularly preferably chlorine, methyl or dimethylamide.

m is 0, 1, 2 or 3. If Z¹ and Z² are cyclopentadienyl derivatives and M is Zr or Hf, then m is preferably 2.

Preference is given to transition metal coordination compounds of the formula (I)

in which

Z¹ is a cyclopentadienyl ligand of the formula (II) or an isomeric form thereof which is produced by irradiation with visible or ultraviolet light and has the formula (IIa)

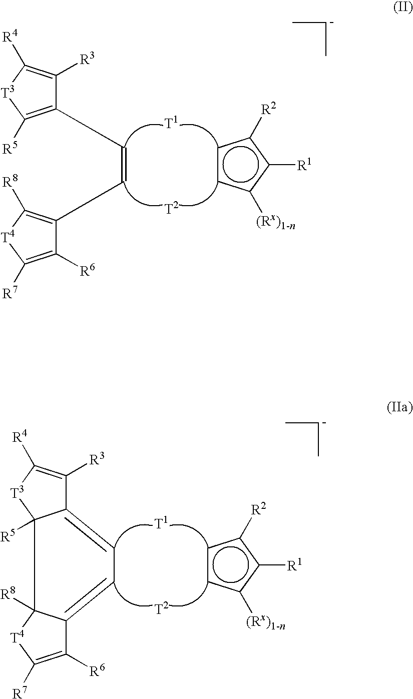

where
R¹ is hydrogen, methyl, ethyl, n-propyl, i-propyl, n-butyl or cyclohexyl, in particular methyl,
R² is hydrogen,
R³, R⁴, R⁶, R⁷ are identical or different and are each hydrogen, fluorine or C₁-C₆-alkyl such as methyl, ethyl or isopropyl or two adjacent radicals R³ and R⁴ and/or R⁶ and R⁷ together with the atoms connecting them form a substituted or unsubstituted, in particular unsubstituted, 1,3-butadiene-1,4-diyl group,
R⁵, R⁸ are identical or different, preferably identical, and are each hydrogen, methyl, hexyl, trifluoromethyl, isopropyl, methoxy or ethoxy, in particular methyl,
R^{x} is hydrogen, methyl, ethyl, n-propyl, i-propyl, n-butyl or cyclohexyl, preferably hydrogen,
T¹ is a single bond and
T² is S,
   A is a bridge between the ligands Z¹ and Z² and is dimethylsilanediyl, diphenylsilanediyl or ethylidene, in particular dimethylsilanediyl,
   Z² is identical to or different from Z¹ and is as defined for Z¹ or is a cyclopentadienyl group which may optionally comprise a 1,2-di(3-thienyl)ethene derivative as photochromic group PCG², where the cyclopentadienyl group is specifically a substituted or unsubstituted cyclopentadienyl radical, a substituted or unsubstituted indenyl radical, a substituted or unsubstituted fluorenyl radical or a substituted or unsubstituted cyclopenta[b]thienylidene radical,
   p is 1,
   n is zero or 1,
   M is Ti, Zr or Hf, in particular Zr or Hf,
   the radicals X are identical or different, preferably identical, and are each chlorine, methyl or dimethylamide, preferably chlorine, and
   m is 2.

The synthesis of the transition metal coordination compounds of the invention is known in principle. It is usual to react a suitable transition metal source, e.g. zirconium tetrachloride, with the desired ligands, e.g. two equivalents of cyclopentadienyl ligand in the form of its lithium salt. To synthesize transition metal coordination compounds having bridged ligands, e.g. bridged cyclopentadienyl ligands, the desired cyclopentadienyl radicals can firstly be joined to one another and subsequently, usually after prior deprotonation, reacted with the transition metal source. WO 03/045964 describes, for example, the synthesis of bridged biscyclopentadienyl metallocenes having two different cyclopentadienyl radicals. In the synthesis of bridged biscyclopentadienyl metallocenes, the resulting transition metal coordination compounds are usually obtained in the form of various diastereomers, which are frequently referred to as rac or pseudo-rac and meso or pseudo-meso (cf. WO 03/045551). The separation of the diastereomers is known in principle.

The above-described synthesis is illustrated by the following example, which does not, however, restrict the scope of the invention, of a preferred transition metal coordination compound of the formula (I), with the synthesis of a preferred ligand precursor Z¹-H likewise being shown.

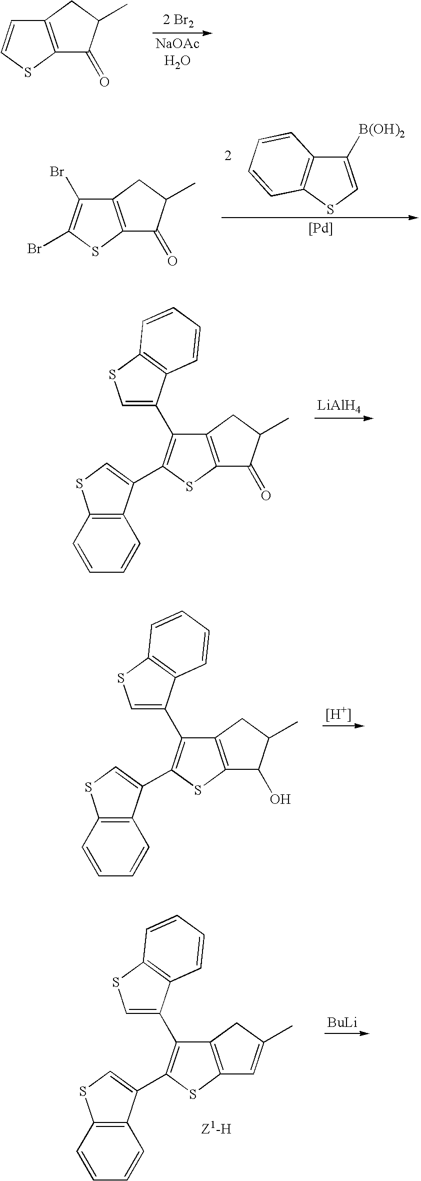

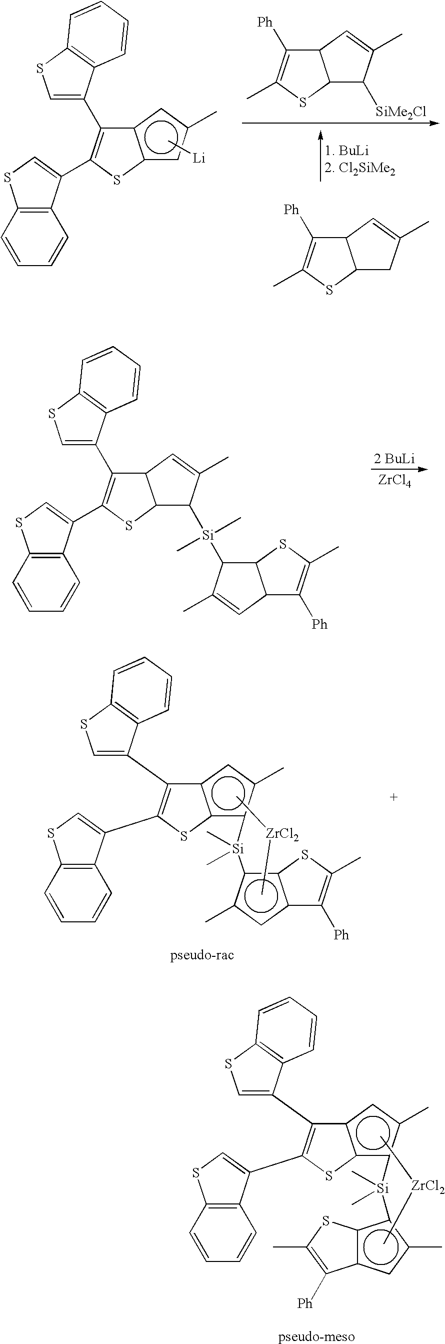

The present invention further provides the compounds of the formulae (III), (IV) and/or (V) or their double bond isomers and/or their photoisomers

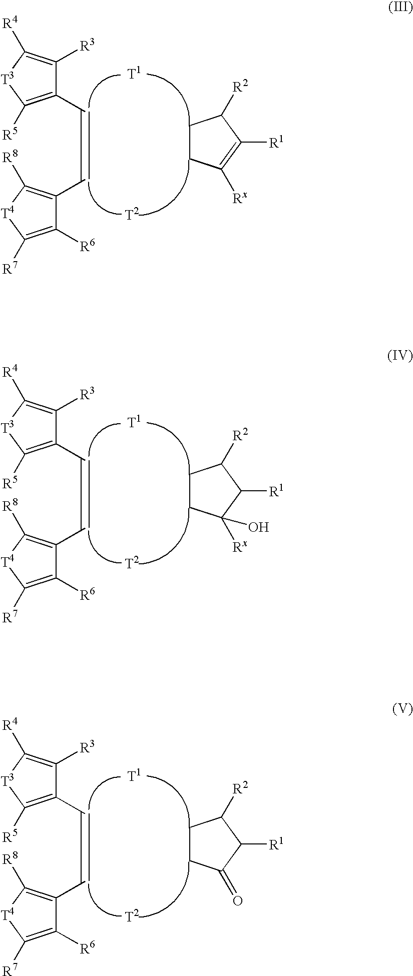

where the variables R¹, R², R³, R⁴, R⁵, R⁶, R⁷, R⁸, R^{X}, T¹, T². T³ and T⁴ are as defined in formula (II).

Particular preference is accordingly given to compounds of the formulae (III), (IV) and (V) or their double bond isomers and/or their photoisomers

in which
R¹ is hydrogen, methyl, ethyl, n-propyl, i-propyl, n-butyl or cyclohexyl, in particular methyl,
R² is hydrogen,
R³, R⁴, R⁶, R⁷ are identical or different and are each hydrogen, fluorine or C₁-C₆-alkyl such as methyl, ethyl or isopropyl or two adjacent radicals R³ and R⁴ and/or R⁶ and R⁷ together with the atoms connecting them form a substituted or unsubstituted, in particular unsubstituted, 1,3-butadiene-1,4-diyl group,
R⁵, R⁶ are identical or different, preferably identical, and are each hydrogen, methyl, hexyl, trifluoromethyl, isopropyl, methoxy or ethoxy, in particular methyl,
R^{x} is hydrogen, methyl, ethyl, n-propyl, i-propyl, n-butyl or cyclohexyl, preferably hydrogen,
T¹ is a single bond and
T², T³, T⁴ are each S.

The novel compounds of the formulae (III), (IV) and (V) are all direct precursors of the particularly preferred cyclopentadienyl ligands Z¹ of the formula (II) or (IIa) which comprises a 1,2-di(3-thienyl)ethene derivative as photochromic group. The photochromic properties of the particularly preferred novel transition metal coordination compounds of the formula (I) are determined by the choice of the corresponding starting compound of the formula (III), (IV) or (V).

The present invention accordingly also provides for the use of a compound of the formula (III) for preparing transition metal coordination compounds.

Starting from a compound of the formula (V), a radical R^{x} which is different from hydrogen can be introduced by reaction of the keto function with, for example, a Grignard compound or a lithium alkyl.

An example of such a reaction is the synthesis of 6-butyl-5-methyl-2,3-bis(benzo[b]thiophen-3-yl)-4,5-dihydrocyclopenta[b]thiophene by reaction of 2,3-di(benzo[b]thiophen-3-yl)-5-methyl-6H-cyclopenta[b]thiophen-6-one with butyllithium and subsequent aqueous work-up, with the tertiary alcohol formed as an intermediate being dehydrated in the work-up.

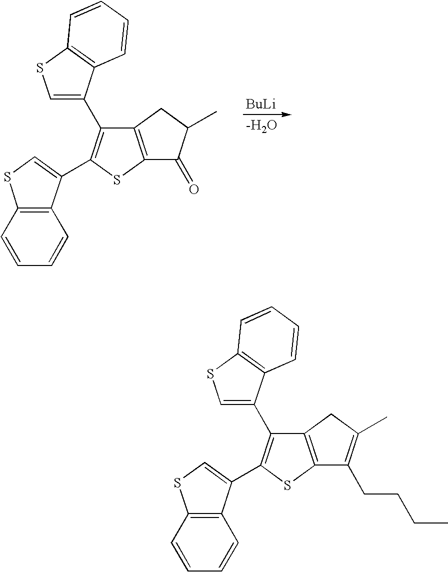

The compounds of the formulae (III), (IV) and (V) can in principle also be used as photochromic compounds for purposes other than the synthesis of transition metal coordination compounds, as described, for example, in Chem. Rev. 2000, 100, 1685-1890. The compounds of the formulae (III), (IV) and (V) are molecular switches and can, for example, also be used as component for preparing polymers or for preparing liquid crystals in order to make possible, in this form, the construction of new optoelectronic components or memory media.

The preferred catalyst systems used in the polymerization process of the invention comprise not only a transition metal coordination compound comprising at least one ligand which is substituted by or fused with at least one photochromic group but also at least one cocatalyst.

The present invention therefore also provides for the use of a transition metal coordination compound comprising at least one ligand which comprises at least one photochromic group for preparing a catalyst system for the polymerization of olefins.

The cocatalyst which together with the transition metal coordination compounds forms a polymerization-active catalyst system is able to convert the transition metal coordination compound into a species which displays polymerization activity toward at least one olefin. The cocatalyst is therefore sometimes also referred to as activating compound. The polymerization-active transition metal species is frequently a cationic species. In this case, the cocatalyst is also frequently referred to as cation-forming compound.

Suitable cocatalysts or cation-forming compounds are, for example, compounds such as an aluminoxane, a strong uncharged Lewis acid, an ionic compound having a Lewis-acid cation or an ionic compound having a Brönsted acid as cation. Preference is given to using an aluminoxane as cocatalyst in the process of the invention.

Particular preference is given to a process according to the invention for preparing olefin polymers, in which the transition metal coordination compound is preactivated by means of an aluminoxane before use in the polymerization reaction. In this preactivation step, the transition metal coordination compounds are, for example as such or as a solution, brought into contact with an aluminoxane, in particular a solution of a methylaluminoxane, for some time, e.g. from 1 minute to 48 hours, preferably from 5 minutes to 4 hours, to form the catalyst system.

In the case of metallocene complexes as transition metal coordination compounds, the cocatalysts are frequently also referred to as metallocenium-ion-forming compounds.

As aluminoxanes, it is possible to use, for example, the compounds described in WO 00/31090. Particularly suitable aluminoxanes are open-chain or cyclic aluminoxane compounds of the general formula (XI) or (XII)

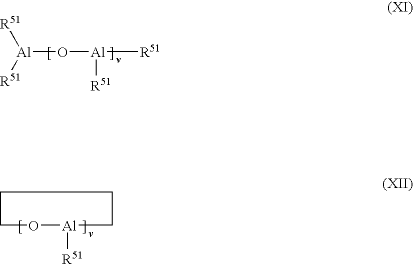

where
R⁵¹ is a C₁-C₄-alkyl group, preferably a methyl or ethyl group, and v is an integer from 30, preferably from 10 to 25.

These oligomeric aluminoxane compounds are usually prepared by reacting a solution of trialkylaluminum with water. In general, the oligomeric aluminoxane compounds obtained in this way are in the form of mixtures of both linear and cyclic chain molecules of various lengths, so that v is to be regarded as a mean. The aluminoxane compounds can also be present in admixture with other metal alkyls, preferably aluminum alkyls.

Furthermore, modified aluminoxanes in which some of the hydrocarbon radicals or hydrogen atoms have been replaced by alkoxy, aryloxy, siloxy or amide radicals can also be used in place of the aluminoxane compounds of the general formula (XI) or (XII).

In the process of the invention, it has been found to be advantageous to use the transition metal coordination compounds and the aluminoxane compounds in such amounts that the atomic ratio of aluminum from the aluminoxane compounds to the transition metal from the transition metal coordination compound is in the range from 1:1 to 100 000:1, preferably in the range from 5:1 to 20 000:1 and in particular in the range from 10:1 to 2000:1.

As strong, uncharged Lewis acids, preference is given to compounds of the general formula (XIII)

M³X¹X²X³ (XIII)

where
M³ is an element of group 13 of the Periodic Table of the Elements, in particular B, Al or Ga, preferably B,
X¹, X² and X³ are each, independently of one another, hydrogen, C₁-C₁₀-alkyl, C₆-C₁₅-aryl, alkylaryl, arylalkyl, haloalkyl or haloaryl each having from 1 to 10 carbon atoms in the alkyl radical and from 6 to 20 carbon atoms in the aryl radical or fluorine, chlorine, bromine or iodine, in particular haloaryl, preferably pentafluorophenyl.

Further examples of strong, uncharged Lewis acids are given in WO 00/31090.

Particular preference is given to compounds of the general formula (XIII), in which X¹, X² and X³ are identical, preferably tris(pentafluorophenyl)borane.

Further strong uncharged Lewis acids which are suitable as cocatalyst or cation-forming compounds are the reaction products from the reaction of a boronic acid with two equivalents of a trialkylaluminum or the reaction products from the reaction of a trialkylaluminum with two equivalents of an acidic fluorinated, in particular perfluorinated, carbon compound such as pentafluorophenol or bis(pentafluorophenyl)borinic acid.

Suitable ionic compounds having Lewis-acid cations include salt-like compounds of the cation of the general formula (XIV)

[(Y^{a+})Q¹Q² . . . Q^{z}]^{d+} (XIV)

where
Y is an element of groups 1 to 16 of the Periodic Table of the Elements,
Q¹, to Q^{z} are singly negatively charged groups such as C₁-C₂₈-alkyl, C₆-C₁₅-aryl, alkylaryl, arylalkyl, haloalkyl, haloaryl each having from 6 to 20 carbon atoms in the aryl radical and from 1 to 28 carbon atoms in the alkyl radical, C₃-C₁₀-cycloalkyl which may optionally bear C₁-C₁₀-alkyl groups as substituents, halogen, C₁-C₂₈-alkoxy, C₆-C₁₅-aryloxy, silyl or mercaptyl groups,
a is an integer from 1 to 6 and
z is an integer from 0 to 5 and
d corresponds to the difference a-z, but d is greater than or equal to 1.

Particularly useful cations are carbonium cations, oxonium cations and sulfonium cations and also cationic transition metal complexes. Particular mention may be made of the triphenylmethyl cation, the silver cation and the 1,1′-dimethylferrocenyl cation. They preferably have noncoordinating counterions, in particular boron compounds as are also mentioned in WO 91/09882, preferably tetrakis(pentafluorophenyl)borate.

Salts having noncoordinating anions can also be prepared by combining a boron or aluminum compound, e.g. an aluminum alkyl, with a further compound which can react to link two or more boron or aluminum atoms, e.g. water, and a third compound which on reaction with the boron or aluminum compound forms an ionizing ionic compound, e.g. triphenylchloromethane. In addition, a fourth compound which likewise reacts with the boron or aluminum compound, e.g. pentafluorophenol, can also be added.

Ionic compounds having Brönsted acids as cations preferably likewise have noncoordinating counterions. As Brönsted acid, particular preference is given to protonated amine or aniline derivatives. Preferred cations are N,N-dimethylanilinium, N,N-dimethylcyclohexylammonium and N,N-dimethylbenzylammonium and also derivatives of the latter two.

Preferred ionic compounds as cocatalysts or cation-forming compounds are, in particular, N,N-dimethylanilinium tetrakis(pentafluorophenyl)borate, N,N-dimethylcyclohexylammonium tetrakis(pentafluorophenyl)borate and N,N-dimethylbenzylammonium tetrakis(pentafluorophenyl) borate.

It is also possible for two or more borate anions to be joined to one another, as in the dianion [(C₆F₅)₂B—C₆F₄—B(C₆F₅)₂]²⁻, or the borate anion can be bound via a bridge having a suitable functional group to the surface of a support particle.

Further suitable cocatalysts or cation-forming compounds are listed in WO 00/31090.

In the process of the invention, the amount of strong, uncharged Lewis acids, ionic compounds having Lewis-acid cations or ionic compounds having Brönsted acids as cations is usually from 0.1 to 20 equivalents, preferably from 1 to 10 equivalents, based on the organic transition metal compound of the formula (I).

Further suitable cocatalysts or cation-forming compounds are boron-aluminum compounds such as di[bis(pentafluorophenyl)boroxy]methylalane. Appropriate boron-aluminum compounds are disclosed, for example, in WO 99/06414.

It is also possible to use mixtures of all the abovementioned cocatalysts or cation-forming compounds. Preferred mixtures comprise aluminoxanes, in particular methylaluminoxane, and an ionic compound, in particular one comprising the tetrakis(pentafluorophenyl)borate anion, and/or a strong uncharged Lewis acid, in particular tris(pentafluorophenyl)borane.

Preference is given to using both the transition metal coordination compounds and the cocatalysts or cation-forming compounds in a solvent, with preference being given to aromatic hydrocarbons having from 6 to 20 carbon atoms, in particular xylenes and toluene.

The catalyst in the process of the invention can further comprise a metal compound of the general formula (XV),

M⁴(R⁵²)ᵣ(R⁵³)ₛ(R⁵⁴)ₜ (XV)

where
M⁴ is an alkali metal, an alkaline earth metal or a metal of group 13 of the Periodic Table, i.e. boron, aluminum, gallium, indium or thallium,
R⁵² is hydrogen, C₁-C₁₀-alkyl, C₆-C₁₅-aryl, alkylaryl or arylalkyl each having from 1 to 10 carbon atoms in the alkyl radical and from 6 to 20 carbon atoms in the aryl radical,
R⁵³ and R⁵⁴ are identical or different and are each hydrogen, halogen, C₁-C₁₀-alkyl, C₆-C₁₅-aryl, alkylaryl, arylalkyl or alkoxy each having from 1 to 10 carbon atoms in the alkyl radical and from 6 to 20 carbon atoms in the aryl radical,
r is an integer from 1 to 3
   and
s and t are integers from 0 to 2, with the sum r+s+t corresponding to the valence of M⁴,
   where the metal compound of the formula (XV) is usually not identical to the cocatalyst or the cation-forming compound. It is also possible to use mixtures of various metal compounds of the formula (XV).

Among the metal compounds of the general formula (XV), preference is given to those in which

M⁴ is lithium, magnesium or aluminum and
R⁵³ and R¹⁴ are each C₁-C₁₀-alkyl.

Particularly preferred metal compounds of the formula (XV) are n-butyllithium, n-butyl-n-octylmagnesium, n-butyl-n-heptylmagnesium, tri-n-hexylaluminum, triisobutylaluminum, triethyllaluminum and trimethylaluminum and mixtures thereof.

If a metal compound of the formula (XV) is used, it is preferably comprised in the catalyst in such an amount that the molar ratio of M⁴ from formula (XV) to the transition metal atom from the transition metal coordination compounds is from 800:1 to 1:1, in particular from 200:1 to 2:1.

The catalyst system comprising a transition metal coordination compound and at least one cocatalyst can, depending on the polymerization process employed, further comprise a support.

To obtain such a supported catalyst system, the unsupported catalyst system can be reacted with a support. The order in which the support, the transition metal coordination compounds and the cocatalyst are combined is in principle immaterial. The transition metal coordination compounds and the cocatalyst can be immobilized independently of one another or simultaneously. After the individual process steps, the solid can be washed with suitable inert solvents such as aliphatic or aromatic hydrocarbons.

As supports, preference is given to using finely divided supports which can be any organic or inorganic, inert solids. In particular, the support can be a porous solid such as talc, a sheet silicate, an inorganic oxide or a finely divided polymer powder (e.g. polyolefin).

Suitable inorganic oxides may be found among oxides of the elements of groups 2, 3, 4, 5, 13, 14, and 16 of the Periodic Table of the Elements. Examples of oxides preferred as supports comprise silicon dioxide, aluminum oxide and also mixed oxides of the elements calcium, aluminum, silicon, magnesium or titanium and also corresponding oxide mixtures. Other inorganic oxides which can be used alone or in combination with the abovementioned preferred oxidic supports are, for example, MgO, ZrO₂, TiO₂ or B₂O₃. A preferred mixed oxide is, for example, calcined hydrotalcite.

The support materials used preferably have a specific surface area in the range from 10 to 1000 m²/g, a pore volume in the range from 0.1 to 5 ml/g and a mean particle size of from 1 to 500 μm. Preference is given to supports having a specific surface area in the range from 50 to 500 m²/g, a pore volume in the range from 0.5 to 3.5 ml/g and a mean particle size in the range from 5 to 350 μm. Particular preference is given to supports having a specific surface area in the range from 200 to 400 m²/g, a pore volume in the range from 0.8 to 3.0 ml/g and a mean particle size of from 10 to 100 μm.

The inorganic support can be subjected to a thermal treatment, e.g. to remove adsorbed water. Such a drying treatment is generally carried out at temperatures in the range from 80 to 300° C., preferably from 100 to 200° C., with drying at from 100 to 200° C. preferably being carried out under reduced pressure and/or a blanket of inert gas (e.g. nitrogen), or the inorganic support can be calcined at temperatures of from 200 to 1000° C. to set, if appropriate, the desired structure of the solid and/or the desired OH concentration on the surface. The support can also be treated chemically, for which purpose it is possible to use customary desiccants such as metal alkyls, preferably aluminum alkyls, chlorosilanes or SiCl₄ or else methylaluminoxane. Appropriate treatment methods are described, for example, in WO 00/31090.

The inorganic support material can also be chemically modified. For example, treatment of silica gel with (NH₄)₂SiF₆ leads to fluorination of the silica gel surface, or treatment of silica gels with silanes comprising nitrogen-, fluorine- or sulfur-comprising groups leads to correspondingly modified silica gel surfaces.

Organic support materials such as finely divided polyolefin powders (e.g. polyethylene, polypropylene or polystyrene) can also be used and should preferably likewise be freed of adhering moisture, solvent residues or other impurities by means of appropriate purification and drying operations before use. It is also possible to use functionalized polymer supports, e.g. ones based on polystyrene, via whose functional groups, for example ammonium or hydroxy groups, at least one of the catalyst components can be immobilized.

In a preferred method of preparation of the supported catalyst system, at least one of the transition metal coordination compounds is brought into contact with at least one cocatalyst as activating or cation-forming compound in a suitable solvent to give a soluble or insoluble, preferably soluble, reaction product, an adduct or a mixture.

The preparation obtained in this way is then mixed with the dehydrated or passivated support material, the solvent is removed and the resulting supported organic transition metal compound catalyst system is dried to ensure that all or most of the solvent is removed from the pores of the support material. The supported catalyst is usually obtained as a free-flowing powder. Examples of the industrial implementation of the above process are described in WO 96/00243, WO 98/40419 or WO 00/05277.

A further preferred embodiment comprises firstly applying the cocatalyst or the cation-forming compound to the support component and subsequently bringing this supported cocatalyst or this cation-forming compound into contact with the transition metal coordination compound.

Further important cocatalyst systems are therefore combinations obtained by combining the following components:
1st component: at least one defined boron or aluminum compound,
2nd component: at least one uncharged compound which has at least one acidic hydrogen atom,
3rd component: at least one support, preferably an inorganic oxidic support, and optionally, as 4th component, a base, preferably an organic nitrogen-comprising base such as an amine, an aniline derivative or a nitrogen heterocycle.

The boron or aluminum compounds used in the preparation of these supported cocatalysts are preferably compounds of the formula (XVI)

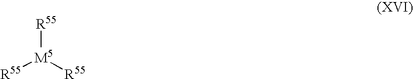

where
the radicals R⁵⁵ are identical or different and are each hydrogen, halogen, C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl or R⁵⁵ is an OSiR⁵⁶₃ group, where
the radicals R⁵⁶ are identical or different and are each hydrogen, halogen, C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, preferably hydrogen, C₁-C₈-alkyl or C₇-C₂₀-arylalkyl, and
M⁵ is boron or aluminum, preferably aluminum.

Particularly preferred compounds of the formula (XVI) are trimethylaluminum, triethylaluminum and triisobutylaluminum.

The uncharged compounds which have at least one acidic hydrogen atom and can react with compounds of the formula (XVI) are preferably compounds of the formulae (XVII), (XVIII) or (XIX),

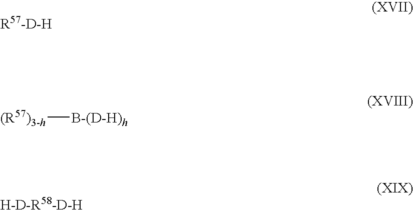

where
the radicals R⁵⁷ are identical or different and are each hydrogen, halogen, a boron-free organic radical having from 1 to 40 carbon atoms, e.g. C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, an Si(R⁵⁹)₃-radical or a CH(SiR⁵⁹₃)₂ radical, where
R⁵⁹ is a boron-free organic radical having from 1 to 40 carbon atoms, e.g. C₁-C₂₀-alkyl, C₁-C₂₀-haloalkyl, C₁-C₁₀-alkoxy, C₆-C₂₀-aryl, C₆-C₂₀-haloaryl, C₆-C₂₀-aryloxy, C₇-C₄₀-arylalkyl, C₇-C₄₀-haloarylalkyl, C₇-C₄₀-alkylaryl, C₇-C₄₀-haloalkylaryl, and
R⁵⁸ is a divalent organic group having from 1 to 40 carbon atoms, e.g. C₁-C₂₀-alkylene, C₁-C₂₀-haloalkylene, C₆-C₂₀-arylene, C₆-C₂₀-haloarylene, C₇-C₄₀-arylalkylene, C₇-C₄₀-haloarylalkylene, C₇-C₄₀-alkylarylene, C₇-C₄₀-haloalkylarylene,
D is an element of group 16 of the Periodic Table of the Elements or an NR⁶⁰ group, where R⁶⁰ is hydrogen or a C₁-C₂₀-hydrocarbon radical such as C₁-C₂₀-alkyl or C₆-C₂₀-aryl, with preference being given to D being oxygen, and
h is 1 or 2.

Suitable compounds of the formula (XVII) are water, alcohols, phenol derivatives, thiophenol derivatives or aniline derivatives, with the halogenated and in particular perfluorinated alcohols and phenols being of particular importance. Examples of particularly suitable compounds are pentafluorophenol, 1,1-bis(pentafluorophenyl)methanol and 4-hydroxy-2,2′,3,3′,4′,5,5′,6,6′-nonafluorobiphenyl.

Suitable compounds of the formula (XVIII) are boronic acids and borinic acids, in particular borinic acids having perfluorinated aryl radicals, for example (C₆F₅)₂BOH.

Suitable compounds of the formula (XIX) are dihydroxy compounds in which the divalent carbon-comprising group is preferably halogenated and in particular perfluorinated. An example of such a compound is 4,4′-dihydroxy-2,2′,3,3′,5,5′,6,6′-octafluorobiphenyl hydrate.

Examples of combinations of compounds of the formula (XVI) with compounds of the formula (XVII) or (XIX) are trimethylaluminum/pentafluorophenol, trimethylaluminum/1-bis(pentafluorophenyl)methanol, trimethylaluminum/4-hydroxy-2,2′,3,3′,4′,5,5′,6,6′-nonafluorobiphenyl, triethyllaluminum/pentafluorophenol or triisobutylaluminum/pentafluorophenol or triethylaluminum/4,4′-dihydroxy-2,2′,3,3′,5,5′,6,6′-octafluorobiphenyl hydrate, with, for example, reaction products of the following type being able to be formed.

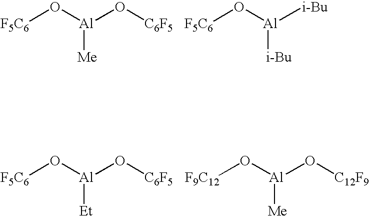

Examples of reaction products from the reaction of at least one compound of the formula (XVI) with at least one compound of the formula (XVIII) are:

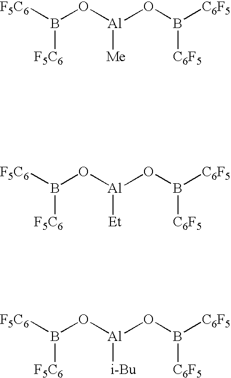

In principle, the components can be combined in any way.

If appropriate, the reaction products from the reaction of at least one compound of the formula (XVI) with at least one compound of the formula (XVII), (XVIII) or (XIX) and optionally the organic nitrogen base are additionally combined with an organometallic compound of the formula (XI), (XII), (XIII) and/or (XV), in order then to form, together with the support, the supported cocatalyst system.

In a preferred embodiment, the 1st component, e.g. compounds of the formula (XII), and the 2nd component, e.g. compounds of the formulae (XVII), (XVIII) or (XIX), are combined and a support as 3rd component and a base as 4th component are combined separately and the two mixtures are subsequently reacted with one another, with the reaction preferably taking place in an inert solvent or suspension medium. The supported cocatalyst formed can be freed of the inert solvent or suspension medium before it is reacted with a transition metal coordination compound and, if appropriate, a metal compound of the formula (XV) to form the catalyst system.

It is also possible firstly to prepolymerize the catalyst solid with α-olefins, preferably linear C₂-C₁₀-1-alkenes and in particular ethylene or propylene, and then to use the resulting prepolymerized catalyst solid in the actual polymerization. The mass ratio of catalyst solid used in the prepolymerization to monomer polymerized onto it is usually in the range from 1:0.1 to 1:200.

Furthermore, a small amount of an olefin, preferably an α-olefin, for example vinylcyclohexane, styrene or phenyldimethylvinylsilane, as modifying component, an antistatic or a suitable inert compound such as a wax or oil can be added as additive during or after the preparation of the supported catalyst system. The molar ratio of additives to transition metal coordination compound is usually from 1:1000 to 1000:1, preferably from 1:5 to 20:1.

In the polymerization process of the invention, the catalyst system is generally used together with a further metal compound of the general formula (XV), which may be different from the metal compound or compounds of the formula (XV) used in the preparation of the catalyst system, for the polymerization or copolymerization of olefins. The further metal compound is generally added to the monomer or the suspension medium and serves to free the monomer of substances which can impair the catalyst activity. It is also possible for one or more further cocatalytic or cation-forming compounds to be additionally added to the catalyst system in the polymerization process.

The polyolefins which can be obtained by the process of the invention for preparing olefin polymers are likewise subject matter of the present invention.

The present invention further provides the catalyst system comprising one or more catalyst constituents, wherein the catalyst system comprises at least one photochromic group in one of the catalyst constituents, which is used in the process of the invention for preparing olefin polymers. The catalyst system can be either a Ziegler-Natta catalyst or a “single site catalyst”. Preference is given to a catalyst system from the class of “single site catalysts”.

The invention therefore further provides a process for preparing a catalyst system for preparing olefin polymers, in which the catalyst constituents are brought into contact with one another and the catalyst system comprises at least one photochromic group in one of the catalyst constituents. The order in which the catalyst constituents are combined is in principle immaterial. However, the catalyst constituents are preferably combined in one of the orders which are described in the literature and have been found to be useful for the respective catalyst type.

Preference is given to a catalyst system of the type described in more detail above which comprises at least one transition metal coordination compound and at least one cocatalyst, wherein the transition metal coordination compound comprises at least one ligand comprising at least one photochromic group. Here, the transition metal coordination compound preferably comprises at least one ligand which is bound to the transition metal center and is selected from the group consisting of cyclopentadienyl derivatives, phenoxyimine derivatives and uncharged or singly or multiply negatively charged bidentate or tridentate nitrogen ligands having two or three coordinating nitrogen atoms.

The invention further provides for the use of the catalyst system of the invention for the polymerization of olefins.

The above-described catalyst system of the invention, which may comprise a support, can be used either alone or together with one or more further catalyst systems which may likewise be supported and are suitable for the homopolymerization, copolymerization or oligomerization of olefins in the polymerization process of the invention. Here, the at least one further catalyst system can be prepared independently of the catalyst system of the invention or can be produced together with this. The different catalyst systems can thus, for example, be present together on a support or they can be present independently of one another as supported or unsupported catalyst systems which can be premixed in any way and thus metered into the polymerization reactor either together or separately and thus independently of one another. The further catalyst system is preferably not able to be changed by light. Examples of known catalyst systems which can be used together with the catalyst system of the invention for preparing polyolefins are, in particular, classical Ziegler-Natta catalysts based on titanium, classical Phillips catalysts based on chromium oxides or single site catalysts which preferably comprise metallocenes, constrained geometry complexes (cf., for example, EP A 0 416 815 or EP A 0 420 436), chromium single site complexes as described, for example, in U.S. Pat. No. 6,437,161, nickel- and palladium-bisimine systems (which can be prepared as described in WO 9803559 A1) or iron- and cobalt-pyridinebisimine compounds (which can be prepared as described in WO 9827124 A1) as transition metal component. If the catalyst system of the invention is used together with at least one further catalyst for the polymerization, then preference is given to using a single site catalyst, in particular one based on an iron-pyridinebisimine compound.

In the process of the invention, the presence of the photochromic group in the catalyst system provides a further means of control for influencing the polymerization process and the polymer produced in a targeted way. This further means of control is independent of the known means of control such as temperature, pressure or hydrogen concentration. Depending on the current state of the photochromic group or the numerical ratio of the two states of the photochromic group, the polymerization properties of the catalyst system are altered.

The invention is illustrated by the following examples which do not, however, restrict the scope of the invention:

### General Procedures:

The synthesis of organic and organometallic compounds involving air sensitive materials were performed under nitrogen atmosphere in pre-dried glassware unless stated otherwise. Manipulations such as filtration and handling, weighing and drying were performed in a Vacuum Atmospheres dri-box with nitrogen flow through arrangement. Solvents used in the synthesis of air-sensitive compounds were Aldrich anhydrous grade. In some cases solvents were further purified by filtration through molecular sieves, activated alumina, or supported copper catalyst in a procedure similarly described by Grubbs et al. in Organometallics 1996, 15(5), 1518-1520.

5-Methyl-4,5-dihydrocyclopenta[b]thiophen-6-one used as starting material was synthesizes according to J. Am. Chem. Soc 1998, 120, 10786-10787.

The palladium catalysts used in the coupling experiments and zirconium-(IV)-chloride was purchased from Strem Chemical Co. (MA, USA); Bis(diphenylphosphino)propane nickel(II) chloride (NiDPPP) was purchased from Aldrich Chemical Company.

Methylalumoxane (solution in toluene; 30% by weight of MAO) used in the polymerizations was procured from Albemarle Corp. and Al(iso-Bu)₃ (1 M solution in toluene) was procured from Aldrich Chemical Company.

Intrinsic Viscosity Number: The viscosity number was determined in decalin at 135° C. in an Ubbelohde viscometer PVS 1 with an S 5 measuring head (both from Lauda). For the sample preparation, 20 mg of polymer were dissolved in 20 ml of decalin at 135° C. over a period of 2 hours. 15 ml of the solution were placed in the viscometer; the instrument carried out a minimum of three running-out time measurements until a consistent result was obtained. The I.V. is calculated from the running-out times in accordance with I.V.=(t/t₀−1)*1/c, where t=mean of the running-out time of the solution, t₀=mean of the running-out time of the solvent, c=concentration of the solution in g/ml.

Gel permeation chromatography: Gel permeation (GPC) was carried out at 145° C. in 1,2,4-trichlorobenzene using a Waters 150C GPC apparatus. The data were evaluated using the software Win-GPC from HS-Entwicklungsgesellschaft für wissenschaftliche Hard-und Software mbH, Ober-Hilbersheim. The columns were calibrated by means of polypropylene standards having molar masses ranging from 100 to 10⁷ g/mol. Mass average (M_{w}) and number average (Mₙ) molar masses of the polymers were determined. The Q value is the ratio of mass average molar mass (M_{w}) to number average molar mass (Mₙ).

Determination of the melting point: The melting point Tₘ was determined by DSC measurement in accordance with ISO Standard 3146 in a first heating phase at a heating rate of 20° C. per minute to 200° C., a dynamic crystallization at a cooling rate of 20° C. per minute down to 25° C. and a second heating phase at a heating rate of 20° C. per minute back to 200° C. The melting point was then the temperature at which the curve of enthalpy versus temperature measured in the second heating phase displayed a maximum.

The density [g/cm³] was determined in accordance with ISO 1183.

The methyl group content was determined by means of IR spectroscopy in analogy with ASTM D 6248-98.

Laser Desorption Ionization-time of flight mass spectra were taken on an Applied Biosystem Voyager System 6033 equipped with a nitrogen laser at 337 nm. Spectra were collected with reflector operations in both positive and negative modes. The acceleration voltage was 20000 V at 76% grid voltage (typical). Mass is reported in either positive mode [M+1] or negative mode [M−1]. Calculated isotope distribution patterns for the parent ion or molecular fragment of the −35 (chlorine) fragments were used to confirm the reported structure.

### EXAMPLES

### 1. {Bis(2,3-(3′-benzothiaphen-yl)]cyclopenta[b]thienyl)ZrCl₂ (1)

### 1a) 2,3-Dibromo-4,5-dihydro-5-methylcyclopenta[b]thiophen-6-one (1a)

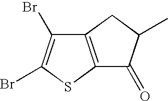

35.7 g (225 mmol) of bromine were added (dropwise) to a mixture of 20.5 g (250 mmol) of sodium acetate, 50 ml of water and 15.2 g (100 mmol) of 5-methyl-6H-cyclopenta[b]thiophen-6-one. After addition was completed, the reaction mixture was stirred for 30 min, sampled (GCMS indicated only one bromine was “on”): an additional 15 g (94 mmol) of bromine were added (dropwise). The reaction mixture was stirred an additional 1 h, then poured into water. Organics were extracted with dichloromethane, washed with a saturated solution of sodium bicarbonate until pH neutral, then water. The organics were dried over magnesium sulphate, filtered and then solvents were removed in vacuo. 29.31 g of a dark purple free flowing solid were collected (95% by GCMS). The material was used in subsequent steps without further purification.

¹H-NMR δ (CDCl₃): 3.2 (m, 1H), 2.98 (m, 1H), 2.57 (m, 1H), 1.3 (t, 3H). EIMS: m/e (%)=312.9 (3), 311.9 (32), 310.9 (10.0), 309.9 (62), 308.9 (11.0), 307.9 (31) 306.9 (4), 296.9 (51), 295.5 (9), 294.9 (100), 292.9

### 1b) 2,3-Di(benzo[b]thiophen-3-yl)-5-methyl-4H-cyclopenta[b]thiophen-6-one (1b)

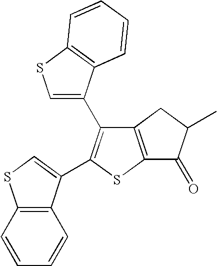

5 g (28 mmol) of thianaptahalene-3 boronic acid (Aldrich) and 2.35 g (22.4 mmol) of sodium carbonate were added to a solution of 1.7 g (5.78 mmol) of 2,3-dibromo-5-methyl-6H-cyclopenta[b]thiophen-6-one dissolved in 70 ml of degassed dimethoxyethane/water (9:1). The flask atmosphere was replaced with nitrogen (further degassing the reaction mixture), then under positive nitrogen flow, 0.7 g (0.6 mmol) of tetrakis-(triphenylphosphine)palladium (Strem, 99.9%) were added. The reaction mixture was refluxed for 2 h and then cooled. The reaction mixture was then poured into a 1N HCl solution, washed with water, saturated bicarbonate solution, then water. The reaction mixture was dried over magnesium sulphate, filtered through silica using 1) hexane, then 2) 50:50 dichloromethane (to collect the product). Solvents were removed in vacuo: 2.03 g (92.8% by GCMS) of the product (1b) were obtained. The product was used in subsequent reactions without further purification.

### Scale-Up Synthesis of (1b)

A solution of 9.3 g (30 mmol) 2,3-dibromocyclopenta[b]thiophen-6-one in 70 ml degassed ethyleneglycol-dimethylether/water (10% water) was placed in a 250 ml flask with side arm and Teflon stirring bar. 25 g (140 mmol) of thianapthalene-3-boronic acid (Aldrich) were added followed by 11.7 g (110 mmol) of sodium carbonate. 2.0 g (2 mmol) of Pd(PPh₃)₄ (Strem) were added, (an additional 30 ml of the DME solution were used to wash in the components); the contents of the flask were then refluxed for 3 h. The mixture was cooled, poured into an iced 1N HCl solution, then the organics were collected with 50:50 dichloromethane/hexane solution. The organics were washed with water, dried over magnesium sulphate, filtered, then solvents removed in vacuo. Solids were then washed with hexane, and then dried. 15.8 g of a pale yellow powder were recovered, 91% product by FIDGC/GCMS. EIMS, m/z (%): 418 (17), 417 (29), 416 (100), 410 (11), 375 (2), 374 (3), 373 (9), 372 (2), 346 (10), 340 (16), 327 (11);

¹H-NMR δ (CDCl₃) (isomers): 7.8 (m, 2H), 7.15-7.55 (m, 8H), 3.5 (2d, 1H), 3.2 (m, 1H), 3.05 (m, 1H), 2.6 (m, 1H), 1.3 (2s, 3H).

### 1c) 6-butyl-5-methyl-2,3-bis(benzo[b]thiophen-3-yl)-4,5-dihydrocyclopenta[b]thiophene (1c)

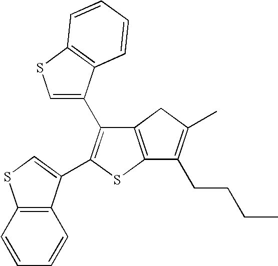

A solution of n-butyllithium (5 mmol, 2 ml, 2.5M in hexane) was added to a solution of 2.08 g (5 mmol) of 2,3-di(benzo[b]thiophen-3-yl)-5-methyl-6H-cyclopenta[b]thiophen-6-one (1b) dissolved in 80 ml of diethyl ether. The reaction mixture was stirred for 18 h and then poured into water. Organics were collected with a 1:1 solution containing dichloromethane and hexane, washed with water, dried over magnesium sulfate, filtered and then solvents were removed in vacuo. In this manner 1.35 g of an orange paste were collected and fractionated by preparative HPLC on 35 g silica using an increasing hexane then dichloromethane gradient. Yield: 0.23 g of compound (1c): 3 isomers by FIDGC/GCMS. EIMS, m/z (%): (major isomer): 459 (5), 458 (19), 457 (33), 456 (100), 454 (1), 430 (10), 430 (10), 429 (16), 429 (16), 428 (29), 427 (93).

¹H-NMR δ (CDCl₃): 7.6-8.0 (m, 3H), 7.6 (d, 1H), 7.65-7.20 (m, 6H), 3.18 (s, 1H), 2.6 (t, 3H), 2.1 (s, 1H), 1.75 (m, 2H), 1.5 (m, 2H), 1.03 (t, 3H).

### 1d) 2,3-di(benzo[b]thiophen-3-yl)-5,6-dihydro-5-methyl-4H-cyclopenta [b]-thiophen-6-ol (1d)

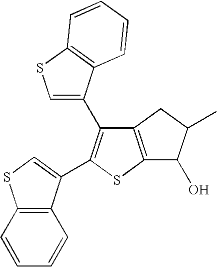

A solution of lithium aluminium hydride (1.8 mmol, 1.8 ml, 1 M in diethyl ether) was added dropwise to a solution of 1.59 g (3.8 mmol) of 3-di(benzo[b]thiophen-3-yl)-5-methyl-6H-cyclopenta[b]thiophen-6-one dissolved in 70 ml of diethyl ether. The reaction mixture was stirred for 1 h at room temperature, and then poured onto ice. The organics were collected with diethyl ether, washed with water, dried over magnesium sulphate, and then filtered. Solvents were removed in vacuo. 1.18 g of (1d) were recovered as light brown powder. GCMS analysis of this material indicated only olefin, which was partially dehydrated by the instrument.

¹H-NMR δ (CDCl₃): 7.7 (m, 2H), 7.45 (m, 1H), 7.2 (4H), 3.4 (m, 0.5H), 3.0-1.9 (7m, 3H), 1.3 (3m, 3H).

### 1e) 2,3-di(benzo[b]thiophen-3-yl)-5-methyl-4H-cyclopenta[b]thiophene (1e)

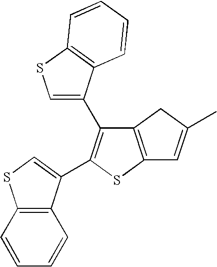

0.6 g of para-toluenesulfonic acid monohydrate were added to a solution of 1.1 g of 2,3-di(benzo[b]thiophen-3-yl)-5,6-dihydro-5-methyl-4H-cyclopenta[b]-thiophen-6-ol (1d) dissolved in 50 ml of toluene. The mixture was refluxed for 1 h, cooled, and then poured into water. The organics were collected with a 50/50 mixture of dichloromethane/hexane, washed with water, dried over magnesium sulphate, filtered and then solvents were removed in vacuo. 1.18 g of a light brown powder were recovered which was then recrystallized from hexane. 0.44 g of (1e) was recovered.

¹H-NMR δ (CDCl₃): 7.9-7.7 (m, 2H), 7.44 (d, 1H), 7.4-7.1 (m, 5H), 3.56 (m, 0.3H), 3.50 (m, 0.3H), 3.5 (s, 0.3), 3.17 (m, 1H), 3.02 (m, 1H), 2.53 (2d, 1H), 1.33 (2s, 3H).

### 1) Bis(2,3-(3′-benzothiaphen-yl)]cyclopenta[b]thienyl)zirconium dichloride (1)

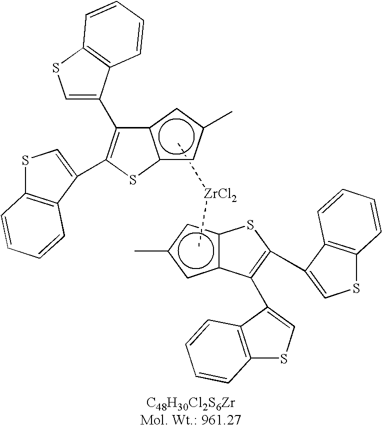

1.04 g (2.5 mmol) of 3-hydro-[2,3-(3′-benzothiaphen-yl)]cyclopenta[b]thiophene (le) slurried in 30 ml of diethyl ether were placed in a 50 ml glass vial with Teflon stirring bar. The solution was irradiated with 254 nm lamps for 90 minutes (color changes from off white to purple during irradiation), then a solution of n-butyllithium was added dropwise. (2.5 mmol, 1 ml, 2.5 M in hexane). The reaction mixture was stirred an additional 1 h, then 0.3 g (1.25 mmol) of zirconium tetrachloride were slowly added as a dry powder. The reaction mixture was stirred overnight, and then filtered. Solids were washed with fresh diethyl ether. The solids were then slurried in dichloromethane, filtered, then solvents removed in vacuo. Finally, the solids recovered from the dichloromethane filtrate were washed with pentane, and then dried in vacuo. Yield of (1): 0.90 g of (1) as an orange free flowing powder.

¹H-NMR δ (CDCl₃): 8.0-6.9 (m, 16H), 6.9-6.1 (4m, 4H), 2.1 (2s, 6H). MS (LD-TOF-negative mode): 922[M⁻-35](loss of —Cl).

### 2) Dimethylsilyl[(5-methyl-2,3-bis(benzothiaphen-3-yl)cyclopenta[b]thien-6-yl)][(2,5-dimethyl-3-phenyl-cyclopenta[b]thien-6-yl)]zirconium dichloride (2)

### 2a) Chloro-dimethyl-(2,5-dimethyl-3-phenylhydrocyclopenta[b]thiophen-6-yl)silane (2a)

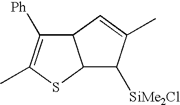

A solution of n-butyllithium (60 mmol, 24 ml, 2.5M in hexene) was added to a solution of 12.7 g (56.2 mmol) of 2,3-dimethyl-3-phenyl-6-hydrocyclopenta[b]thiophene dissolved in 100 ml of diethyl ether. Additional 100 ml of diethyl ether were added to facilitate stirring. The reaction mixture was stirred for 5 h becoming a white slurry.

In a separate flask, a solution containing 10.9 g (86 mmol) of dichlorodimethylsilane dissolved in 50 ml of diethyl ether was prepared. The contents from the flask above were added via cannula, stirred for 18 h, then solvents removed in vacuo. The solids were slurried in dichloromethane, then filtered. Solvents were removed from the filtrate: 6.37 g of a dark brown viscous oil were recovered. EIMS, m/z (%): [M] 318, (100%).

### 2b) Dimethyl-[5-methyl-[(2,3-bis(benzothiaphen-3-yl)hydrocyclopenta[b]thiophen-6-yl)][(2,5-dimethyl-3-phenylhydrocyclopenta[b]thiophen-6-yl)]silane (isomers) (2b)

1.04 g (2.5 mmol) of (2,3-bis(benzothiaphen-3-yl)cyclopenta[b]-thiophene were dissolved in 80 ml of diethyl ether. The solution was stirred under 254 nm UV light for 90 min. Then a solution containing n-butyl lithium was added (2.5 mmol, 1 ml, 2.5M in hexane). The reaction mixture was stirred for 2 h, then a solution containing 0.82 g (2.6 mmol) of chlorodimethyl-(2,5-dimethyl-3-phenylcyclopenta[b]thiophen-6-yl)]silane dissolved in diethyl ether was added slowly. The reaction mixture was stirred for 72 h and then poured into water. Organics were collected with a 20% dichloromethane/hexane solution, the organics were washed with water, dried over magnesium sulfate, filtered, the solvents removed in vacuo. 1.75 g of the product were filtered (HPLC) through silica gel (Isco column, 110 g) using hexane and dichloromethane gradient. In this fashion, 0.21 g of (2b) were recovered as a light pink free flowing powder.

¹H-NMR δ (CDCl₃): 8.0-7.6 (m, 5H), 7.6-7.0 (m, 10H), 6.6-6.4 (2s, 2H), 4.2-3.8 (1d, 2s, 2H), 2.5 (s, 3H), 2.5 (m, 6H), −0.5 (m, 6H); LCMS, [M]=683.

### 2) Dimethylsilyl[(5-methyl-(2,3-bis(benzothiaphen-3-yl)cyclopenta[b]thien-6-yl)][(2,5-dimethyl-3-phenyl-cyclopenta[b]thien-6-yl)]zirconium dichloride (2)

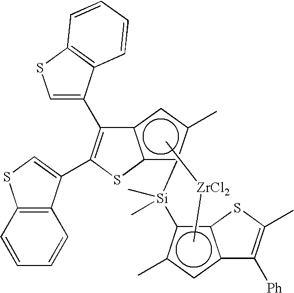

A solution of 0.18 g (0.26 mmol) of dimethyl-[5-methyl-[(2,3-benzothiaphen-3-yl)cyclopenta[b]-thiophen-6-yl][(2,5-dimethyl-3-phenylthiophen-yl)cyclopenta[b]thiophen-6-yl]silane (2b) in 40 ml of diethyl ether was irradiated for 4 h with two 254 nm lamps. Under irradiation, a solution containing n-butyllithium was added (0.6 mmol, 0.25 ml, 2.5 M in hexane). The reaction mixture was stirred for 18 h, then 0.6 g (0.26 mmol) of zirconium tetrachloride were added as a dry powder. The reaction mixture was stirred for 18 h, then filtered. Solvents were removed from the yellow ethereal solution and solids remaining were dried in vacuo: 0.34 g.

¹H-NMR δ (CDCl₃): 7.9 (d, 0.5H), 7.8 (d, 0.5H), 7.74 (t, 2H), 7.5 (t, 2H), 7.46-7.1 (m, 10H), 7.0 (m, 1H), 6.6, 2s, 1H), 6.5 (2s, 1H), 2.5-2.3 (m, 6H), 1.1 (m, 6H), 0.05 (s, 3H).

MS (LD-TOF-positive mode): 843[M⁺+1]

### Example P1

### Solution Polymerization of Propene

20 ml of toluene and a solution containing MAO (2.8 mmol, 0.9 ml, 1.58 M in toluene) were placed in a 30 ml Schlenk tube equipped with a Teflon stirring bar. In a separate vial, the metallocene (1) was weighed and a solution made with 5 ml toluene and an additional amount of MAO (2.8 mmol, 0.9 ml, 1.58 M in toluene). The color change was noted and the solutions were combined. Propylene was bubbled through the solution at a rate of about 15 to 25 I/h and the time recorded. The polymerization reaction mixture was poured into an acidic methanol solution; the polymer was collected by filtration, washed with acetone and dried in vacuo. This gave 0.12 g of polypropylene. Results of the polymerization and results of the polymer analysis are shown in table 1 below.

### Example P2

### Solution Polymerization of Propene with 254 nm UV

In a manner identical to example P1 above, 20 ml of toluene and a solution containing MAO (2.8 mmol, 0.9 ml, 1.58 M in toluene) were placed in a 30 ml Schlenk tube equipped with a Teflon stirring bar. In a separate vial, the metallocene (1) was weighed and a solution made with 5 ml toluene and an additional amount of MAO (2.8 mmol, 0.9 ml, 1.58 M in toluene). The color change was noted and the solutions were combined. Two laboratory low wattage 254 nm UV lamps were turned on and placed on either side of the Schlenk tube during the duration of the experiment. Propylene was bubbled through the solution at a rate of about 15 to 25 l/h and the time recorded. The polymerization reaction mixture was poured into an acidic methanol solution; the polymer was precipitated with acetone and the polymer collected by filtration. This gave 0.09 g of polypropylene Results of the polymerization and results of the polymer analysis are shown in table 1 below.

### Example P3

### Solution Polymerization of Propene

In a manner identical to P1 the polymerization reactor and solutions were prepared. The amount of metallocene (1) was increased to 10 mg and the polymerization time extended to 90 min. This gave 0.38 g of polypropylene. Results of the polymerization and results of the polymer analysis are shown in table 1 below.

### Example P4

### Solution Polymerization of Propene with 254 nm UV

In a manner identical to P2 the polymerization reactor and solutions were prepared. The amount of metallocene (1) was increased to 10 mg and the polymerization time in the presence of 254 nm UV irradiation extended to 90 min. This gave 0.04 g of polypropylene Results of the polymerization and results of the polymer analysis are shown in table 1 below.

TABLE 1
Propylene solution polymerizations comparing without (P1, P3)
and with (P2, P4) 254 nm UV irradiation during polymerization.

| | | | | | Activity | | |
|---|---|---|---|---|---|---|---|
| | MC | | time | yield PP | [g PP/(mmol | M_{w} | |
| Example | [mg] | Al/Zr | (min) | [g] | cat * h)] | [g/mol] | Q |
| P1 | 6 | 427 | 60 | 0.12 | 18 | 29015 | 3.3 |
| P2 | 5 | 570 | 60 | 0.09 | 18 | 21427 | 5.2 |
| P3 | 10 | 532 | 90 | 0.38 | 24 | 14311 | 3.4 |
| P4 | 10 | 532 | 90 | 0.04 | 3 | 7293 | 4.3 |

### Example P5

### Solution Co-Polymerization of Ethylene and Hexene

A solution containing 5.2 mg metallocene (2) and MAO (0.6 ml of a 30% toluene solution) was added to a mechanically stirred 11 glass reaction flask confining 250 ml of toluene and 5 ml of hexene. Ethylene gas was bubbled through the solution at a rate of 30 l/h. The temperature was maintained at 40° C. and the time was recorded. At the end of the polymerization, the reaction flask was purged with argon, then a solution containing 50 ml of methanol and 30 ml of an aqueous, 1N HCl solution was added. The polymer was collected by filtration, washed with methanol, and then dried: This gave 1.1 g of polyethylene-hexene copolymer. Results of the polymerization and results of the polymer analysis are shown in table 2 below.

### Example P6

### Solution Co-Polymerization of Ethylene and Hexene with 254 nm UV

In an experiment carried out exactly identical to P5, the reactor and 5.3 mg metallocene (2) were prepared and added to the polymerization reactor. Two laboratory low wattage 254 nm UV lamps were turned on and placed on either side of the reaction flask during the duration of the experiment. The polymer was collected in an identical fashion as in P5: This gave 6.5 g of polyethylene-hexene copolymer. Results of the polymerization and results of the polymer analysis are shown in table 2 below.

TABLE 2
Toluene solutiolen ethylene and ethylene-hexene polymerization
results with (2)

Example

| | P5 | P6 |
|---|---|---|
| MC (1) | 5.2 | 5.3 |
| [mg] | | |
| hexane | 5 | 5 |
| [ml] | | |
| Polymerization time | 20 | 20 |
| [min] | | |
| Yield | 1.1 | 6.5 |
| [g_{Polymer}] | | |
| Activity | 535 | 3100 |
| [kg_{Polymer}/(mol_{Zr} * h)] | | |
| Tₘ2 | 79 | 86 |
| [° C.] | | |
| ΔH2 | 45 | 48 |
| [J/g] | | |
| IV | 3.8 | 3.4 |
| [dl/g] | | |
| IR-Density | 0.8953 | 0.885 |
| [g/cm³] | | |
| IR-% hexene | ~16 | ~20 |
| IR-CH₃ | ~22 | 36.5 |
| [1/1000 C.] | | |
| M_{w} | 436 | 433 |
| [10⁻³ g/mol] | | |
| Q | 6.4 | 4.0 |

1. A process for preparing olefin polymers comprising polymerizing or copolymerizing at least one olefin of the formula R^{a}—CH═CH—R^{b}, where R^{a} and R^{b} are identical or different and are each a hydrogen atom or a hydrocarbon radical having from 1 to 20 carbon atoms, or R^{a} and R^{b} together with the atoms connecting them can form a ring, at a temperature of from 60 to 200° C. and a pressure of from 0.5 to 100 bar, in solution, in suspension or in the gas phase in the presence of a catalyst system comprising at least one catalyst constituent, wherein the catalyst system comprises at least one photochromic group in one of the catalyst constituents.

2. The process according to claim 1, wherein the photochromic group is selected from the group consisting of 1,2-diarylethene derivatives, 1,2-diheteroarylethene derivatives, dihydropyrene derivatives, fulgides, acetanilides, aldehyde hydrazones, thioindigo derivatives, rhodamine derivatives, spiropyrans, spirooxazines, azobenzene derivatives and anthraquinone derivatives.

3. The process according to claim 1, wherein the polymerization further comprises transforming the at least one photochromic group from a state A into a state B or from the state B into the state A in the presence of at least one light source.

4. The process according to claim 1, wherein the catalyst system further comprises at least one transition metal coordination compound and, optionally a cocatalyst, with the transition metal coordination compound comprising at least one ligand which is substituted by or fused with at least one photochromic group.

5. The process according to claim 4, wherein the transition metal coordination compound comprises at least one ligand selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms which is bound to the transition metal center and is substituted by or fused with at least one photochromic group.

6. A catalyst system for preparing olefin polymers which comprises at least one catalytic constituent wherein the catalyst system comprises at least one photochromic group in one of the catalyst constituents.

7. The catalyst system according to claim 6 further comprising at least one transition metal coordination compound and at least one cocatalyst, wherein the transition metal coordination compound comprises at least one ligand comprising at least one photochromic group.

8. A process for preparing a catalyst system for the preparation of olefin polymers, comprising bringing catalytic constituents into contact with one another, wherein the catalyst system comprises at least one photochromic group in one of the catalyst constituents.

9. A transition metal coordination compound comprising a transition metal and a transition metal coordination compound, wherein the transition metal is selected from the group of elements consisting of Sc, Yb, La, Ti, Zr, Hf, V, Nb, Ta, Mo, W, Fe, Co, Ni, Pd and the lanthanides, and the transition metal coordination compound comprises at least one ligand comprising at least one photochromic group.

10. A transition metal coordination compound comprising a transition metal and a transition metal coordination compound, wherein the transition metal is selected from the group of elements consisting of Sc, Yb, La, Ti, Zr, Hf, Cr, V, Nb, Ta, Mo, W, Fe, Co, Ni, Pd and the lanthanides, and the transition metal coordination compound comprises at least one ligand selected from the group consisting of cyclopentadienyl derivatives, phenoxyimine derivatives and uncharged or singly or multiply negatively charged monodentate, bidentate or tridentate nitrogen ligands having one, two or three coordinating nitrogen atoms which is substituted by or fused with at least one photochromic group.

11. A compound of the formula (III), (IV) or (V) 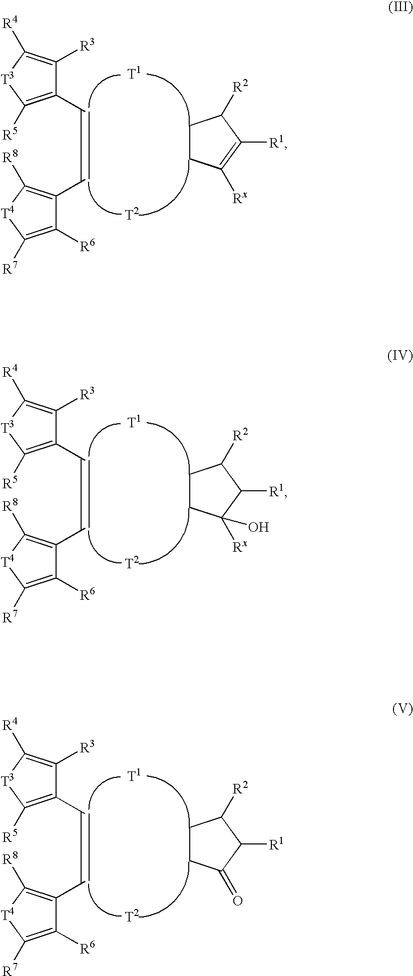
where
R¹ is hydrogen or an organic radical having from 1 to 40 carbon atoms;
R² is hydrogen or an organic radical having from 1 to 40 carbon atoms;
R³, R⁴, R⁶, R⁷ are identical or different and are each hydrogen, halogen or an organic radical having from 1 to 40 carbon atoms or two adjacent radicals R³ and R⁴ and/or R⁶ and R⁷ together with the atoms connecting them form a monocyclic or polycyclic, substituted or unsubstituted, aliphatic or aromatic ring system which has from 4 to 40 carbon atoms and can also comprise heteroatoms selected from the group consisting of the elements Si, Ge, N, P, O, S, Se and Te;
R⁵, R⁸ are identical or different, and are each hydrogen or an organic radical having from 1 to 40 carbon atoms;
R^{x} is hydrogen or an organic radical having from 1 to 40 carbon atoms;
T¹ is a single bond or a divalent organic group having from 1 to 40 carbon atoms;
T² is O, S, Se, Te, NR⁹, PR⁹ or CR⁹₂, where the radicals R⁹ are identical or different and are each hydrogen or an organic radical having from 1 to 40 carbon atoms; and
T³, T⁴ are identical or different and are each O, S, Se, Te, NR⁹ or PR⁹,
or a double bond isomer thereof and/or a photoisomer thereof.

12. The compound of claim 11 wherein R² is hydrogen.

13. The compound of claim 11 wherein R⁵ and R⁸ are identical.
